(19) United States
(12) Patent Application Publication
(10) Pub. No.: US 2017/0235366 A1
(43) Pub. Date: Aug. 17, 2017

(54) DOMINANT LIMB IDENTIFICATION METHOD AND DEVICE

(71) Applicant: BEIJING ZHIGU TECH CO., LTD., BEIJING (CN)

(72) Inventor: HAO LIU, BEIJING (CN)

(21) Appl. No.: 15/501,766

(22) PCT Filed: Aug. 7, 2015
(86) PCT No.: PCT/CN2015/086310

§ 371 Date: Feb. 3, 2017

(30) Foreign Application Priority Data
Aug. 7, 2014 (CN) 201410386783.1
Aug. 26, 2014 (CN) 201410426333.0
Sep. 4, 2014 (CN) 201410447649.8
Nov. 27, 2014 (CN) 201410705591.2
Nov. 27, 2014 (CN) 201410705598.4
Nov. 27, 2014 (CN) 201410705808.X
Nov. 27, 2014 (CN) 201410705907.8
Nov. 27, 2014 (CN) 201410705910.X
Nov. 27, 2014 (CN) 201410708930.2
Nov. 27, 2014 (CN) 201410708988.7
Nov. 27, 2014 (CN) 201410710049.6

(51) Int. Cl. G06F 3/01; A61B 5/026; A61B 5/00; A61B 5/01; A61B 5/145; A61B 5/0402; A61B 5/11; A61B 5/024

(52) U.S. Cl. CPC G06F 3/015; A61B 5/11; A61B 5/026; A61B 5/02416; A61B 5/01; A61B 5/14539; A61B 5/0402; A61B 5/14542; A61B 5/4872; A61B 5/0531

(57) ABSTRACT

The present application relates to the field of wearable devices and provides a dominant limb identification method and device. The method may comprise acquiring first somatosensory information of a first limb of a user and determining whether the first limb is a dominant limb according to the first somatosensory information and reference information. The method and device enable a device worn by the user to perform automatic setting according to the identification result, thereby improving user experience.

### CROSS-REFERENCE TO RELATED APPLICATIONS

The present international patent cooperative treaty (PCT) application claims the benefit of priority to Chinese Patent Application No. 201410386783.1, which was filed on Aug. 7, 2014, and entitled “Dominant Limb Identification Method and Device”; Chinese Patent Application No. 201410426333.0, which was filed on Aug. 26, 2014, and entitled “Dominant Limb Identification Method and Device”; Chinese Patent Application No. 201410447649.8, which was filed on Sep. 4, 2014, and entitled “Dominant Limb Identification Method and Device”; and eight Chinese Patent Application Nos. 201410705591.2, 201410710049.6, 201410705910.X, 201410705808.X, 201410705907.8, 201410705598.4, 201410708930.2, and 201410708988.7, which were all filed on Nov. 27, 2014, and entitled “Dominant Limb Determining Method and Device.” Each of the above-identified applications is hereby incorporated into the present international PCT application by reference in its entirety.

### TECHNICAL FIELD

The present application relates to the field of wearable devices, and in particular, to a dominant limb identification method and device.

### BACKGROUND

In recent years, with the development of wearable devices, smart wristbands, smart bracelets and smart eyeglasses have gradually been entering into people's daily life, making the daily life more convenient and colorful. Wearable devices generally have limited interaction capability due to their small volume. Therefore, users hope to have a wearable device with good self-identifying capability, so as to reduce the number of setting operations that needs to be performed by them.

About 10-13% of the population is left-hand dominant, and the others are right-hand dominant. If a wearable device can identify the dominant hand of a user, the identification result can be used as an input to the wearable device or another device, so as to reduce the number of setting operations that needs to be performed by the user, thereby improving user experience.

### SUMMARY

An aspect of the present application involves providing a dominant limb identification method and device.

According to one aspect of the present disclosure, a dominant limb identification method is provided. The method may comprise acquiring first somatosensory information of a first limb of a user and determining whether the first limb is a dominant limb according to the first somatosensory information and reference information.

According to another aspect of the present disclosure, a dominant limb identification device is provided. The device may comprise a first acquiring module configured to acquire first somatosensory information of a first limb of a user. The device may also comprise a first determining module configured to identify whether the first limb is a dominant limb according to the first somatosensory information and reference information.

The disclosed method and device for determining a dominant limb enable a device worn by the user to perform automatic setting according to the identification result, thereby improving user experience.

### BRIEF DESCRIPTION OF THE DRAWINGS

FIG. 1 is a flowchart of an exemplary dominant limb identification method, according to some embodiments of the present application;

FIG. 2a is a curve showing exemplary acceleration information of a dominant hand of a user;

FIG. 2b is a curve showing exemplary acceleration information of a non-dominant hand of a user;

FIG. 3 is a flowchart of an exemplary method implementing step S140, according to an embodiment of the present application;

FIG. 4 is a flowchart of another exemplary method implementing step S140, according to an embodiment of the present application;

FIG. 5 shows an exemplary comparison between blood volumes of wrist arteries of a dominant hand and a non-dominant hand of a user;

FIG. 6 is a flowchart of an exemplary method implementing step S140, according to an embodiment of the present application;

FIG. 7 is a flowchart of another exemplary method implementing step S140, according to an embodiment of the present application;

FIG. 8 shows an exemplary comparison between PPG signals of a dominant hand and a non-dominant hand of a user;

FIG. 9 is a flowchart of an exemplary method implementing step S140, according to an embodiment of the present application;

FIG. 10 is a flowchart of another exemplary method implementing step S140, according to an embodiment of the present application;

FIG. 11 shows an exemplary comparison between temperature signals of a dominant hand and a non-dominant hand of a user;

FIG. 12 is a flowchart of an exemplary method implementing step S140, according to an embodiment of the present application;

FIG. 13 is a flowchart of another exemplary method implementing step S140, according to an embodiment of the present application;

FIG. 14 shows an exemplary comparison between humidity signals of a dominant hand and a non-dominant hand of a user;

FIG. 15 is a flowchart of an exemplary method implementing step S140, according to an embodiment of the present application;

FIG. 16 is a flowchart of another exemplary method implementing step S140, according to an embodiment of the present application;

FIG. 17 shows an exemplary comparison between fat ratio information of a dominant hand and a non-dominant hand of a user;

FIG. 18 is a flowchart of an exemplary method implementing step S140, according to an embodiment of the present application;

FIG. 19 is a flowchart of another exemplary method implementing step S140, according to an embodiment of the present application;

FIG. 20 shows an exemplary comparison between alkalinity/acidity information (PH value) of a dominant hand and a non-dominant hand of a user;

FIG. 21 is a flowchart of an exemplary method implementing step S140, according to an embodiment of the present application;

FIG. 22 is a flowchart of another exemplary method implementing step S140, according to an embodiment of the present application;

FIG. 23 shows an exemplary comparison between skin conductance information of a dominant hand and a non-dominant hand of a user;

FIG. 24 is a flowchart of an exemplary method implementing step S140, according to an embodiment of the present application;

FIG. 25 is a flowchart of another exemplary method implementing step S140, according to an embodiment of the present application;

FIG. 26 is a flowchart of another exemplary method implementing step S140, according to an embodiment of the present application;

FIG. 27 is a flowchart of an exemplary method implementing step S140, according to an embodiment of the present application;

FIG. 28 is a flowchart of another exemplary method implementing step S140, according to an embodiment of the present application;

FIG. 29 shows an exemplary comparison between bio-impedance signals of a dominant hand and a non-dominant hand of a user;

FIG. 30 is a flowchart of an exemplary method implementing step S140, according to an embodiment of the present application;

FIG. 31 is a flowchart of another exemplary method implementing step S140, according to an embodiment of the present application;

FIG. 32 shows an exemplary comparison between blood oxygen information of a dominant hand and a non-dominant hand of a user;

FIG. 33 is a flowchart of an exemplary method implementing step S140, according to an embodiment of the present application;

FIG. 34 is a flowchart of another exemplary method implementing step S140, according to an embodiment of the present application;

FIG. 35 is a flowchart of another exemplary dominant limb identification method, according to some embodiments of the present application;

FIG. 36 is a flowchart of another exemplary dominant limb identification method, according to some embodiments of the present application;

FIG. 37 is a flowchart of another exemplary dominant limb identification method, according to some embodiments of the present application;

FIG. 38 is a flowchart of another exemplary dominant limb identification method, according to some embodiments of the present application;

FIG. 39 is a block diagram of an exemplary dominant limb identification device, according to some embodiments of the present application;

FIG. 40 is a block diagram of another exemplary dominant limb identification device, according to some embodiments of the present application;

FIG. 41 is a block diagram of another exemplary dominant limb identification device, according to some embodiments of the present application;

FIG. 42 is a block diagram of another exemplary dominant limb identification device, according to some embodiments of the present application;

FIG. 43 is a block diagram of another exemplary dominant limb identification device, according to some embodiments of the present application;

FIG. 44 is a block diagram of another exemplary dominant limb identification device, according to some embodiments of the present application;

FIG. 45 is a block diagram of another exemplary dominant limb identification device, according to some embodiments of the present application;

FIG. 46 is a block diagram of another exemplary dominant limb identification device, according to some embodiments of the present application;

FIG. 47 is a block diagram of another exemplary dominant limb identification device, according to some embodiments of the present application;

FIG. 48 is a block diagram of another exemplary dominant limb identification device, according to some embodiments of the present application;

FIG. 49 is a block diagram of another exemplary dominant limb identification device, according to some embodiments of the present application;

FIG. 50 is a block diagram of another exemplary dominant limb identification device, according to some embodiments of the present application;

FIG. 51 is a block diagram of another exemplary dominant limb identification device, according to some embodiments of the present application;

FIG. 52 is a block diagram of another exemplary dominant limb identification device, according to some embodiments of the present application;

FIG. 53 is a block diagram of another exemplary dominant limb identification device, according to some embodiments of the present application;

FIG. 54 is a block diagram of another exemplary dominant limb identification device, according to some embodiments of the present application;

FIG. 55 is a block diagram of another exemplary dominant limb identification device, according to some embodiments of the present application;

FIG. 56 is a block diagram of another exemplary dominant limb identification device, according to some embodiments of the present application;

FIG. 57 is a block diagram of another exemplary dominant limb identification device, according to some embodiments of the present application;

FIG. 58 is a block diagram of another exemplary dominant limb identification device, according to some embodiments of the present application;

FIG. 59 is a block diagram of another exemplary dominant limb identification device, according to some embodiments of the present application;

FIG. 60 is a block diagram of another exemplary dominant limb identification device, according to some embodiments of the present application;

FIG. 61 is a block diagram of another exemplary dominant limb identification device, according to some embodiments of the present application;

FIG. 62 is a block diagram of another exemplary dominant limb identification device, according to some embodiments of the present application;

FIG. 63 is a block diagram of another exemplary dominant limb identification device, according to some embodiments of the present application;

FIG. 64 is a block diagram of another exemplary dominant limb identification device, according to some embodiments of the present application;

FIG. 65 is a block diagram of another exemplary dominant limb identification device, according to some embodiments of the present application; and

FIG. 66 is a schematic diagram of an exemplary hardware structure of a dominant limb identification device, according to some embodiments of the present application.

### DETAILED DESCRIPTION

Embodiments of the present application are described in further detail below with reference to the accompanying drawings. The following embodiments are intended to describe the present application, but not to limit the scope of the present application.

It should be understood by a person skilled in the art that in the embodiments of the present application, the value of the serial number of each step described below does not mean an execution sequence, and the execution sequence of each step should be determined according to the function and internal logic thereof, and should not be construed as any limitation on the implementation procedure of the embodiments of the present application.

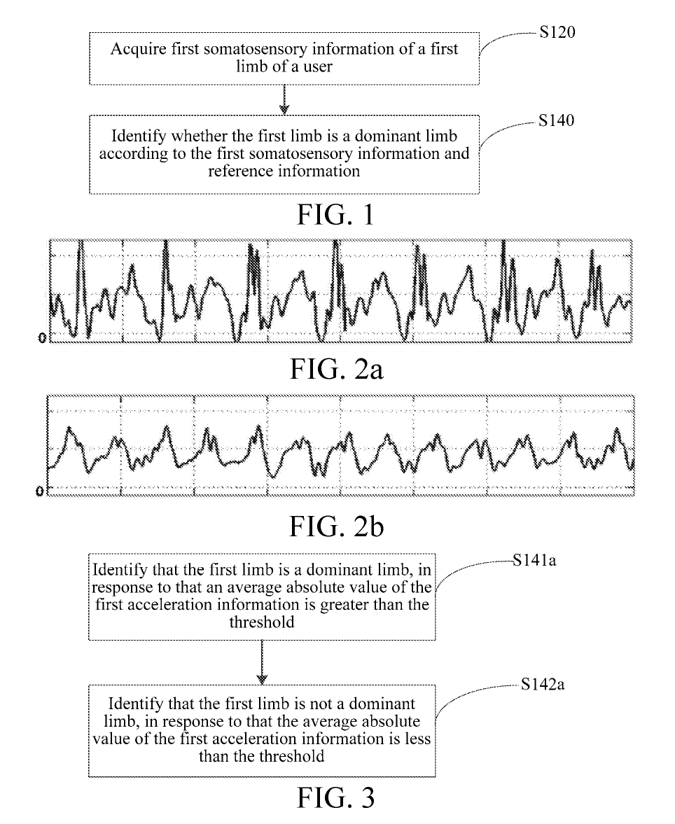

FIG. 1 is a flowchart of an exemplary dominant limb identification method according to one embodiment of the present application, where the method may be implemented on, for example, a dominant limb identification device. As shown in FIG. 1, the method may comprise the following steps:

S120: acquiring first somatosensory information of a first limb of a user; and

S140: determining whether the first limb is a dominant limb according to the first somatosensory information and reference information.

In the method according to this embodiment of the present application, first somatosensory information of a first limb of a user is acquired, and it is determined whether the first limb is a dominant limb according to the first somatosensory information and reference information. In this way, a method for determining a dominant limb is provided, which enables a device worn by the user to perform automatic setting according to the determination result, thereby improving user experience.

The functions of the steps S120 and S140 will be described in detail below with reference to specific implementation manners.

S120: Acquire first somatosensory information of a first limb of a user.

The first limb may be one of the two hands of the user or one of the two arms of the user. For simplicity, an example in which the first limb is one of the two hands of the user is mainly described below.

The first somatosensory information may be acceleration information, blood flow information, PPG information, temperature information, humidity information, fat information, alkalinity/acidity information, skin conductance information, electromyogram information, bio-impedance information or blood oxygen information of the first limb, which may be collected/acquired by using a corresponding sensor. For example, the acceleration information of the first limb may be collected/acquired by using an acceleration sensor.

S140: Determine whether the first limb is a dominant limb according to the first somatosensory information and reference information.

a) The first somatosensory information may be acceleration information of the first limb, e.g., first acceleration information. The step S140 may be:

S140a: determining whether the first limb is a dominant limb according to the first acceleration information and reference information.

The first acceleration information may be acceleration values in a period of time, which may be acquired by using an acceleration sensor worn on the first limb according to a predetermined sampling frequency. For example, the acceleration sensor may perform sampling at a frequency of 10 Hz, and obtain 6000 sampled values within 10 minutes. The acceleration value may comprise coordinate components on three coordinate axes X, Y, and Z. For example, assume that an acceleration value corresponding to one sampling point is (2, −5, 10), indicating that the coordinate component on the X axis is 2, the coordinate component on the Y axis is −5, and the coordinate component on the Z axis is 10.

A dominant limb of a person may be used more frequently than a non-dominant limb, in other words, a dominant limb of a person may move more frequently than a non-dominant limb. Therefore, after acceleration information of a dominant limb and a non-dominant limb of a user are sampled at a same sampling frequency within a period of time (for example, 10 minutes), it can be found that an average absolute value of the acceleration information of the dominant limb is higher than an average absolute value of the acceleration information of the non-dominant limb.

FIG. 2a is a curve showing acceleration information of a dominant hand of a user, where a transverse axis represents a sampling time, and a longitudinal axis represents an absolute acceleration value; FIG. 2b is a curve showing acceleration information of a non-dominant hand of the user within the same period of time, where a transverse axis represents a sampling time, and a longitudinal axis represents an absolute acceleration value. It can be seen that, within a same period of time, a sum of absolute acceleration values of the dominant hand is greater than a sum of absolute acceleration values of the non-dominant hand at all sampling points, and correspondingly, an average absolute value of the acceleration information of the dominant hand is greater than an average absolute value of the acceleration information of the non-dominant hand within this period of time.

The average absolute value of the acceleration information refers to an average value of absolute values of the acceleration information. For example, assume that the acceleration information comprises acceleration values at the following three sampling points: a first sampling point (2, −5, 10), a second sampling point (12, −15, 5), and a third sampling point (4, 6, 8). In this case, it can be obtained that the absolute value of an acceleration corresponding to the first sampling point is 11.36, the absolute value of an acceleration corresponding to the second sampling point is 19.85, and the absolute value of an acceleration corresponding to the third sampling point is 10.77. Therefore, the average absolute value of the acceleration information is 13.99.

In the present application, dominant limb identification can be achieved based on the above principle.

In one implementation manner, the reference information may be a threshold determined according to an average absolute value of left-limb acceleration information and an average absolute value of right-limb acceleration information of the user.

Referring to FIG. 3, the step S140a may comprise:

S141a: determining that the first limb is a dominant limb, in response to that an average absolute value of the first acceleration information is greater than the threshold; and

S142a: determining that the first limb is not a dominant limb, in response to that the average absolute value of the first acceleration information is less than the threshold.

For example, the reference information may be a threshold determined according to an average absolute value of left-hand acceleration information and an average absolute value of right-hand acceleration information of the user. Assuming that the average absolute value of the left-hand acceleration information of the user falls within a first range (Lₘᵢₙ, Lₘₐₓ), assuming that the average absolute value of the right-hand acceleration information of the user falls within a second range (Rₘᵢₙ, Rₘₐₓ), and assuming that the left hand of the user is a dominant hand, it is obtained that Lₘᵢₙ>Rₘₐₓ, and it can be determined that the threshold is M, and Lₘₙ>M>Rₘₐₓ. That is, the threshold M is a value between the first range and the second range.

Therefore, if the average absolute value of the first acceleration information is greater than the threshold M, it is considered that it falls within the first range, and the first limb is a dominant hand; if the average absolute value of the first acceleration information is less than the threshold M, it is considered that it falls within the second range, and the first limb is not a dominant hand.

It should be noted that, in this implementation manner, the reference information needs to be determined according to the left-limb acceleration information and the right-limb acceleration information of the user; therefore, the left-limb acceleration information and the right-limb acceleration information of the user need to be acquired in advance, for example, before formally using the dominant limb identification device, the user wears the device on the left hand for a period of time and then wears the device on the right hand for a period of time, so as to complete a training process.

In addition, to ensure the accuracy of identification, a sampling time for the first acceleration information should be long enough, for example, greater than a time threshold (for example, 10 minutes).

In another implementation manner, the reference information may be second acceleration information of a second limb of the user.

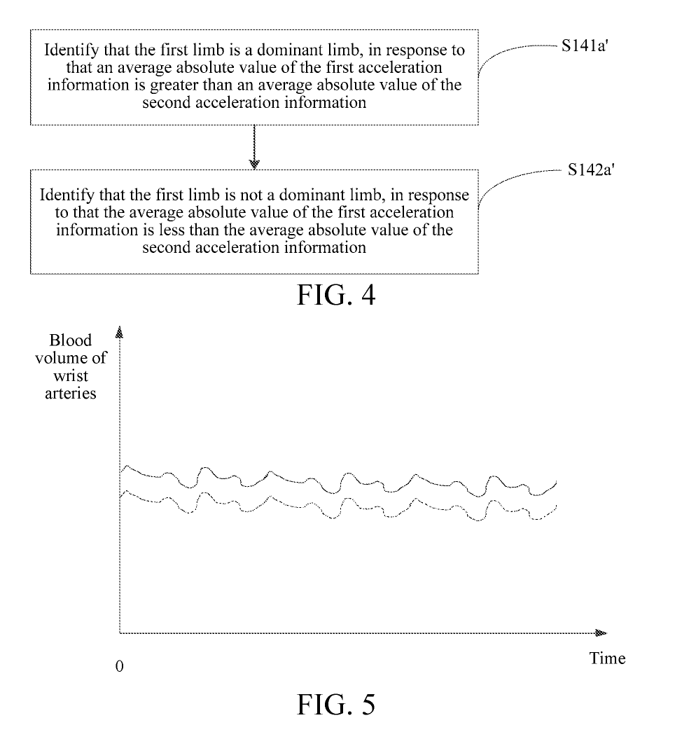

Referring to FIG. 4, the step S140a may comprise:

S141a′: determining that the first limb is a dominant limb, in response to that an average absolute value of the first acceleration information is greater than an average absolute value of the second acceleration information; and

S142a′: determining that the first limb is not a dominant limb, in response to that the average absolute value of the first acceleration information is less than the average absolute value of the second acceleration information.

In this implementation manner, the user does not need to perform training in advance, and may, for example, separately acquire the first acceleration information of the first limb and the second acceleration information of the second limb of the user, and then compare the average absolute value of the first acceleration information with the average absolute value of the second acceleration information, so as to identify a dominant limb.

This implementation manner is advantageous in that the user does not need to complete a training process intentionally, and collection of the reference information is completed during normal use by the user; however, the method in this implementation manner may require acquiring acceleration information of, for example, both hands, and is mainly applicable to scenarios in which the user needs to move the two hands at the same time, for example, the user wears smart gloves on the two hands to play an electronic game.

b) The first somatosensory information may be blood flow information of the first limb, e.g., first blood flow information. The step S140 may be:

S140b: determining whether the first limb is a dominant limb according to the first blood flow information and reference information.

The first blood flow information is blood flow information acquired on the first limb. The first blood flow information may be information about a blood flow rate of artery vessels at the wrist or ankle over a period of time, e.g., the volume of blood that flows through artery vessels at the wrist or ankle per unit time. The first blood flow information may be acquired by using at least one blood volume measuring sensor that is close to or in contact with the skin of the user, and the blood volume measuring sensor may be, for example, an ultrasonic sensor or a PhotoPlethysmoGraphy (PPG) sensor.

The left ventricle contracts and pumps the blood into the aorta, then the blood flows through various levels of arteries to capillary networks in all parts of the body, and in a case in which the left ventricle contracts and generates a pressure, because wrist artery vessels of a dominant limb are larger than wrist artery vessels of a non-dominant limb, a volume of blood that flows through the wrist artery vessels of the dominant limb per unit time may be greater than a volume of blood that flows through the wrist artery vessels of the non-dominant limb per unit time.

FIG. 5 is a schematic diagram of comparison between blood volumes of wrist arteries of a dominant hand and a non-dominant hand of a user, where the abscissa represents time, the ordinate represents a blood volume, a solid curve represents a blood flow information curve of the dominant hand, and a dashed curve represents a blood flow information curve of the non-dominant hand. It can be seen that, the blood volume of wrist arteries of the dominant hand is generally higher than the blood volume of wrist arteries of the non-dominant hand. The blood volume of wrist arteries refers to a total volume of blood that flows through wrist arteries per minute, and is one of the most important basic indicators of the blood circulation function of the human body. The cardiac output can be further calculated according to the blood volume of wrist arteries. The cardiac output is an important indicator for assessment of the efficiency of the circulatory system.

The inventor has also found that a difference similar to that shown in FIG. 5 also exists between blood flow information of other limbs (for example, foot or leg) of the user. Determination of a dominant limb can be implemented based on the above principle.

In one implementation manner, the reference information is second blood flow information of a second limb of the user. The method may further comprise:

S130b: acquiring the second blood flow information of the second limb of the user as the reference information.

For example, two sets of blood volume measuring sensors may be disposed, to collect blood flow information of the first limb and the second limb of the user at the same time, and the blood flow information collected on the second limb, e.g., the second blood flow information, is used as the reference information.

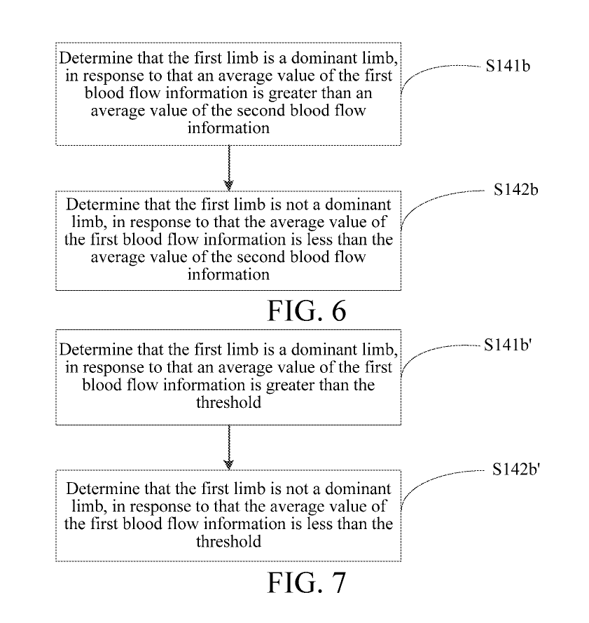

In this implementation manner, in the step S140, it may be determined whether the first limb is a dominant limb by comparing an average value of the first blood flow information with an average value of the second blood flow information. The average value of the first blood flow information is an average value of blood flow rates corresponding to multiple sampling time points in the first blood flow information, and similarly, the average value of the second blood flow information is an average value of blood flow rates corresponding to multiple sampling time points in the second blood flow information. The use of the average value can avoid the problem that a sampling error occurring at a single sampling time point leads to incorrect determination of a dominant limb, thereby improving the accuracy of dominant limb determination. Specifically, as shown in FIG. 6, the step S140b may comprise:

S141b: determining that the first limb is a dominant limb, in response to that an average value of the first blood flow information is greater than an average value of the second blood flow information; and

S142b: determining that the first limb is not a dominant limb, in response to that the average value of the first blood flow information is less than the average value of the second blood flow information.

In another implementation manner, the reference information may be a threshold determined according to an average value of left-limb blood flow information and an average value of right-limb blood flow information of the user. Specifically, as shown in FIG. 7, the step S140b may comprise:

S141b′: determining that the first limb is a dominant limb, in response to that an average value of the first blood flow information is greater than the threshold; and

S142b′: determining that the first limb is not a dominant limb, in response to that the average value of the first blood flow information is less than the threshold.

For example, the right-limb blood flow information and the left-limb blood flow information of the user are collected in advance, analyzed and processed. Assuming that the average value of the right-limb blood flow information falls within a first range

R_{bmin}, R_{bmax}), assuming that the average value of the left-limb blood flow information falls within a second range (L_{bmin}, L_{bmax}), and assuming that the right limb is a dominant limb, it is obtained that L_{bmax}<R_{bmin}, and it can be determined that the threshold is M_{b}, and L_{bmax}<M_{b}<R_{bmin}. That is, the threshold M_{b} is a value between the first range and the second range.

Therefore, if the average value of the first blood flow information is greater than the threshold M_{b}, it is considered that it falls within the first range, and the first limb is a dominant limb of the user; if the average value of the first blood flow information is less than the threshold M_{b}, it is considered that it falls within the second range, and the first limb is not a dominant limb of the user.

Generally, an average value of blood flow information of a dominant limb is at least 5% higher than an average value of blood flow information of a non-dominant limb, based on which the threshold M_{b} can be set appropriately.

c) The first somatosensory information may be PPG information of the first limb, e.g., first PPG information. The step S140 may be:

S140c: determining whether the first limb is a dominant limb according to the first PPG information and reference information.

The first PPG information may be PPG information acquired on the first limb. The first PPG information may be PPG signals in a period of time, and may be acquired by using at least one PPG sensor that is close to or in contact with the skin of the user. The PPG signal may be a voltage change signal that reflects a change in blood volume caused by throbbing of artery vessels and is obtained by a photodetector when light is transmitted or reflected to pass through blood vessels in the human body.

The inventor has found that because a dominant limb of a person is larger and stronger than a non-dominant limb and has rougher skin and thicker skin layer than the non-dominant limb, the transmittance of skin of the non-dominant limb is, for example, lower than the transmittance of skin of the dominant limb, and therefore, an amplitude value of PPG information collected on the non-dominant limb may be higher than an amplitude value of PPG information collected on the dominant limb.

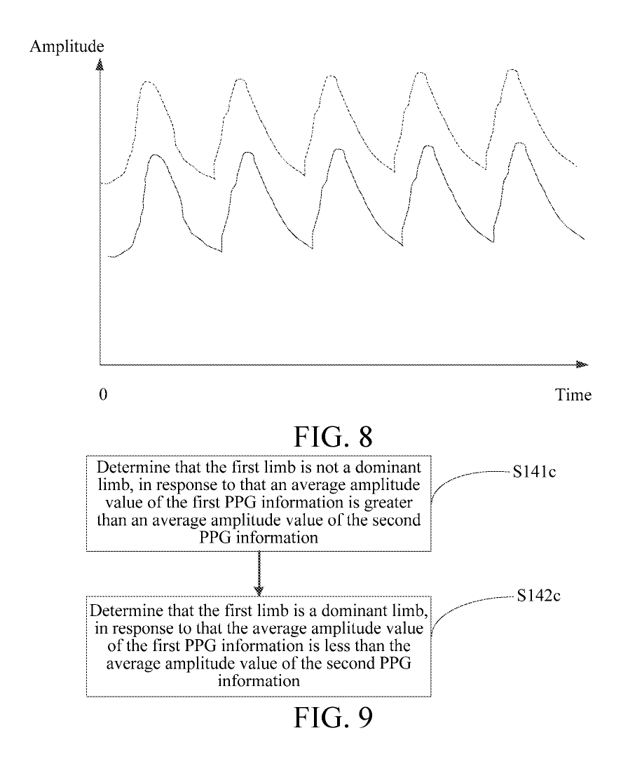

FIG. 8 is a schematic diagram of comparison between PPG signals of a dominant hand and a non-dominant hand of a user, where the abscissa represents time, the ordinate represents an amplitude value of a PPG signal, a solid curve represents a PPG signal curve of the dominant hand, and a dashed curve represents a PPG signal curve of the non-dominant hand. It can be seen that, the amplitude value of the PPG signal of the non-dominant hand is generally higher than the amplitude value of the PPG signal of the dominant hand. The amplitude value in the present application is an amplitude of a waveform corresponding to the PPG signal, and its value is non-negative.

The inventor has also found that a difference similar to that shown in FIG. 8 also exists between PPG signals of other limbs (for example, arm, foot or leg) of the user. Determination of a dominant limb can be implemented based on the above principle.

In one implementation manner, the reference information is second PPG information of a second limb of the user. The method may further comprise:

S130c: acquiring the second PPG information of the second limb of the user as the reference information.

For example, two sets of PPG sensors may be disposed, to collect PPG information of the first limb and the second limb of the user at the same time, and the PPG information collected on the second limb, e.g., the second PPG information, is used as the reference information.

In this implementation manner, in the step S140c, it may be determined whether the first limb is a dominant limb by comparing an average amplitude value of the first PPG information with an average amplitude value of the second PPG information. The average amplitude value of the first PPG information is an average value of PPG amplitude values corresponding to multiple sampling points in the first PPG information, and similarly, the average amplitude value of the second PPG information is an average value of PPG amplitude values corresponding to multiple sampling points in the second PPG information. The use of the average value can avoid the problem that a sampling error occurring at a single sampling time point leads to incorrect determination of a dominant limb, thereby improving the accuracy of dominant limb determination. Specifically, as shown in FIG. 9, the step S140c may comprise:

S141c: determining that the first limb is not a dominant limb, in response to that an average amplitude value of the first PPG information is greater than an average amplitude value of the second PPG information; and

S142c: determining that the first limb is a dominant limb, in response to that the average amplitude value of the first PPG information is less than the average amplitude value of the second PPG information.

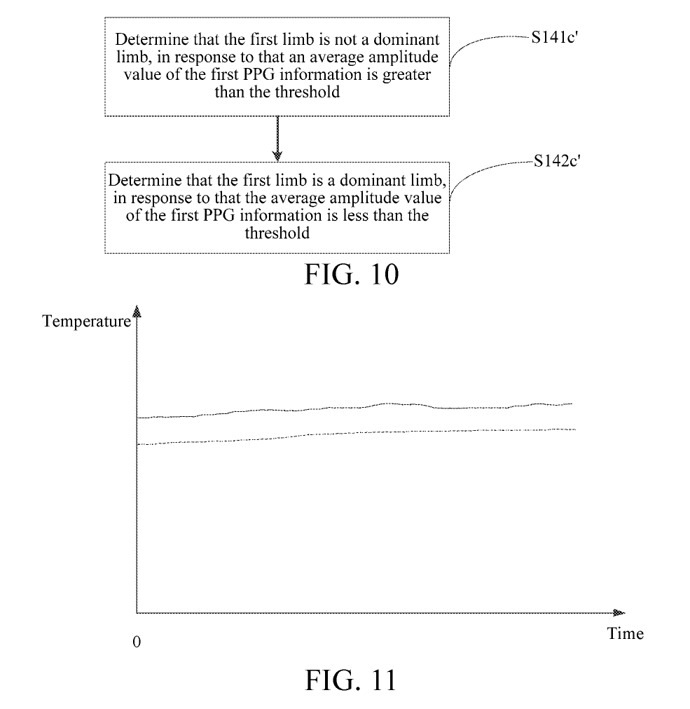

In another implementation manner, the reference information may be a threshold according to an average amplitude value of left-limb PPG information and an average amplitude value of right-limb PPG information of the user. Specifically, as shown in FIG. 10, the step S140c may comprise:

S141c′: determining that the first limb is not a dominant limb, in response to that an average amplitude value of the first PPG information is greater than the threshold; and

S142c′: determining that the first limb is a dominant limb, in response to that the average amplitude value of the first PPG information is less than the threshold.

For example, the right-limb PPG information and the left-limb PPG information of the user are collected in advance, analyzed and processed. Assuming that the average amplitude value of the right-limb PPG information falls within a first range (Rₚₘᵢₙ, Rₚₘₐₓ), assuming that the average amplitude value of the left-limb PPG information falls within a second range (Lₚₘᵢₙ, Lₚₘₐₓ), and assuming that the left limb is a dominant limb, it is obtained that Lₚₘₐₓ<Rₚₘᵢₙ, and it can be determined that the threshold is Mₚ, and Lₚₘₐₓ<Mₚ<Rₚₘᵢₙ. That is, the threshold Mₚ is a value between the first range and the second range.

Therefore, if the average amplitude value of the first PPG information is greater than the threshold Mₚ, it is considered that it falls within the first range, and the first limb is not a dominant limb of the user; if the average amplitude value of the first PPG information is less than the threshold Mₚ, it is considered that it falls within the second range, and the first limb is a dominant limb of the user.

Generally, an average amplitude value of PPG information of a non-dominant limb is at least 5% higher than an amplitude average value of PPG information of a dominant limb, based on which the threshold Mₚ can be set appropriately.

d) The first somatosensory information may be temperature information of the first limb, e.g., first temperature information. The step S140 may be:

S140d: determining whether the first limb is a dominant limb according to the first temperature information and reference information.

The first temperature information may be temperature information acquired on the first limb. The first temperature information may be temperature signals in a period of time, and may be acquired by using at least one temperature sensor that is close to or in contact with the skin of the user.

The inventor has found that because blood vessels of a dominant limb of a person are generally larger than blood vessels of a non-dominant limb and the dominant limb moves more frequently than the non-dominant limb and generates more heat, the temperature of the dominant limb may be higher than the temperature of the non-dominant limb.

FIG. 11 is a schematic diagram of comparison between temperature signals of a dominant hand and a non-dominant hand of a user, where the abscissa represents time, the ordinate represents the temperature value, a solid curve represents a temperature signal curve of the dominant hand, and a dashed curve represents a temperature signal curve of the non-dominant hand. It can be seen that the temperature value of the dominant hand is generally higher than the temperature value of the non-dominant hand.

The inventor has also found that a difference similar to that shown in FIG. 11 also exists between temperature signals of other limbs (for example, arm, foot or leg) of the user. Determination of a dominant limb can be implemented based on the above principle.

In one implementation manner, the reference information is second temperature information of a second limb of the user. The method may further comprise:

S130d: acquiring the second temperature information of the second limb of the user as the reference information.

For example, two sets of temperature sensors may be disposed, to collect temperature information of the first limb and the second limb of the user at the same time, and the temperature information collected on the second limb, e.g., the second temperature information, is used as the reference information.

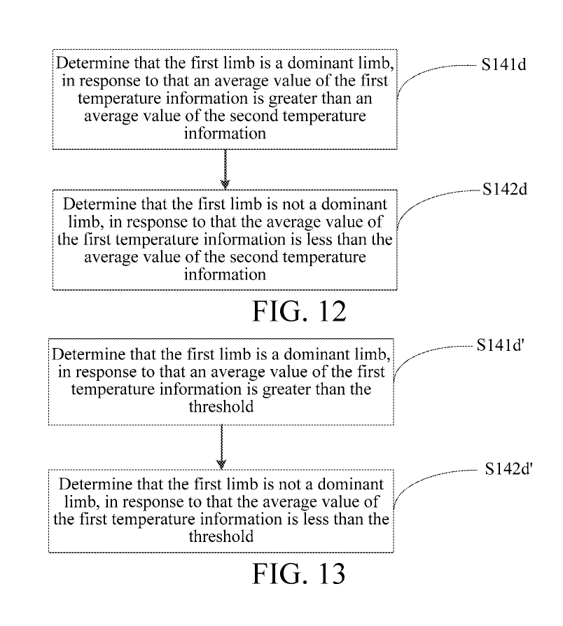

In this implementation manner, in the step S140d, it may be determined whether the first limb is a dominant limb by comparing an average value of the first temperature information with an average value of the second temperature information. The average value of the first temperature information is an average value of temperature values corresponding to multiple sampling time points in the first temperature information, and similarly, the average value of the second temperature information is an average value of temperature values corresponding to multiple sampling time points in the second temperature information. The use of the average value can avoid the problem that a sampling error occurring at a single sampling time point leads to incorrect determination of a dominant limb, thereby improving the accuracy of dominant limb determination. Specifically, as shown in FIG. 12, the step S140d may comprise:

S141d: determining that the first limb is a dominant limb, in response to that an average value of the first temperature information is greater than an average value of the second temperature information; and

S142d: determining that the first limb is not a dominant limb, in response to that the average value of the first temperature information is less than the average value of the second temperature information.

In another implementation manner, the reference information may be a threshold determined according to an average value of left-limb temperature information and an average value of right-limb temperature information of the user. Specifically, as shown in FIG. 13, the step S140d may comprise:

S141d′: determining that the first limb is a dominant limb, in response to that an average value of the first temperature information is greater than the threshold; and

S142d′: determining that the first limb is not a dominant limb, in response to that the average value of the first temperature information is less than the threshold.

For example, the right-limb temperature information and the left-limb temperature information of the user are collected in advance, analyzed and processed. Assuming that the average value of the right-limb temperature information falls within a first range (Rₜₘᵢₙ, Rₜₘₐₓ), assuming that the average value of the left-limb temperature information falls within a second range (Lₜₘᵢₙ, Lₜₘₐₓ), and assuming that the right limb is a dominant limb, it is obtained that Lₜₘₐₓ<Rₜₘᵢₙ, and it can be determined that the threshold is Mₜ, and Lₜₘₐₓ<Mₜ<Rₜₘᵢₙ. That is, the threshold Mₜ is a value between the first range and the second range.

Therefore, if the average value of the first temperature information is greater than the threshold Mₜ, it is considered that it falls within the first range, and the first limb is a dominant limb of the user; if the average value of the first temperature information is less than the threshold Mₜ, it is considered that it falls within the second range, and the first limb is not a dominant limb of the user.

Generally, an average value of temperature information of a dominant limb is at least 0.75 Celsius degrees higher than an average value of temperature information of a non-dominant limb, based on which the threshold Mₜ can be set appropriately.

e) The first somatosensory information may be humidity information of the first limb, e.g., first humidity information. The step S140 may be:

S140e: determining whether the first limb is a dominant limb according to the first humidity information and reference information.

The first humidity information may be humidity information acquired on the first limb. The first humidity information may be humidity signals in a period of time, and may be acquired by using at least one humidity sensor that is close to or in contact with the skin of the user.

The inventor has found that because sweat glands of a dominant limb of a person are more well-developed than sweat glands of a non-dominant limb and the dominant limb moves more frequently than the non-dominant limb and generates more heat to stimulate sweat glands to produce sweat, the humidity of the dominant limb is higher than the humidity of the non-dominant limb.

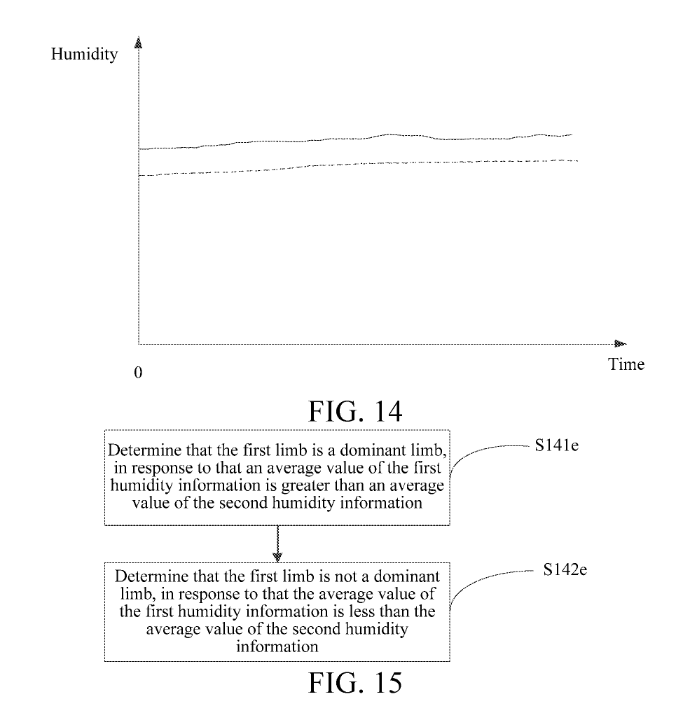

FIG. 14 is a schematic diagram of comparison between humidity signals of a dominant hand and a non-dominant hand of a user, where the abscissa represents time, the ordinate represents a humidity value, a solid curve represents a humidity signal curve of the dominant hand, and a dashed curve represents a humidity signal curve of the non-dominant hand. It can be seen that, the humidity value of the dominant hand is generally higher than the humidity value of the non-dominant hand.

The inventor has also found that a difference similar to that shown in FIG. 14 also exists between humidity signals of other limbs (for example, arm, foot or leg) of the user. Determination of a dominant limb can be implemented based on the above principle.

In one implementation manner, the reference information is second humidity information of a second limb of the user. The method may further comprise:

S130e: acquiring the second humidity information of the second limb of the user as the reference information.

For example, two sets of humidity sensors may be disposed, to collect humidity information of the first limb and the second limb of the user at the same time, and the humidity information collected on the second limb, e.g., the second humidity information, is used as the reference information.

In this implementation manner, in the step S140e, it may be determined whether the first limb is a dominant limb by comparing an average value of the first humidity information with an average value of the second humidity information. The average value of the first humidity information is an average value of humidity values corresponding to multiple sampling time points in the first humidity information, and similarly, the average value of the second humidity information is an average value of humidity values corresponding to multiple sampling time points in the second humidity information. The use of the average value can avoid the problem that a sampling error occurring at a single sampling time point leads to incorrect determination of a dominant limb, thereby improving the accuracy of dominant limb determination. Specifically, as shown in FIG. 15, the step S140e may comprise:

S141e: determining that the first limb is a dominant limb, in response to that an average value of the first humidity information is greater than an average value of the second humidity information; and

S142e: determining that the first limb is not a dominant limb, in response to that the average value of the first humidity information is less than the average value of the second humidity information.

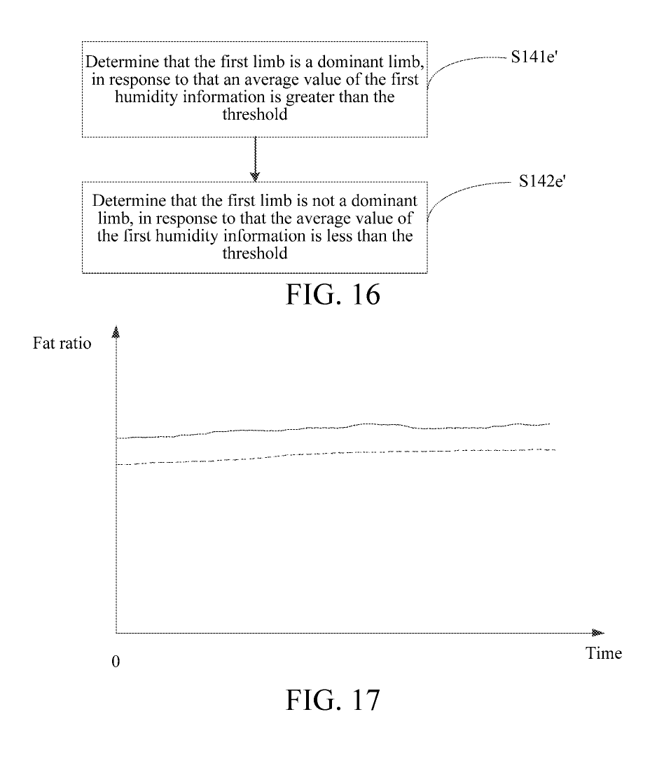

In another implementation manner, the reference information may be a threshold determined according to an average value of left-limb humidity information and an average value of right-limb humidity information of the user. Specifically, as shown in FIG. 16, the step S140e may comprise:

S141e′: determining that the first limb is a dominant limb, in response to that an average value of the first humidity information is greater than the threshold; and

S142e′: determining that the first limb is not a dominant limb, in response to that the average value of the first humidity information is less than the threshold.

For example, the right-limb humidity information and the left-limb humidity information of the user are collected in advance, analyzed and processed. Assuming that the average value of the right-limb humidity information falls within a first range (Rₕₘᵢₙ, Rₕₘₐₓ), assuming that the average value of the left-limb humidity information falls within a second range (Lₕₘᵢₙ, Lₕₘₐₓ), and assuming that the right limb is a dominant limb, it is obtained that Lₕₘₐₓ<Rₕₘᵢₙ, and it can be determined that the threshold is Mₕ, and Lₕₘₐₓ<Mₕ<Rₕₘᵢₙ. That is, the threshold Mₕ is a value between the first range and the second range.

Therefore, if the average value of the first humidity information is greater than the threshold Mₕ, it is considered that it falls within the first range, and the first limb is a dominant limb of the user; if the average value of the first humidity information is less than the threshold Mₕ, it is considered that it falls within the second range, and the first limb is not a dominant limb of the user.

Generally, an average value of humidity information of a dominant limb is at least 3% higher than an average value of humidity information of a non-dominant limb, based on which the threshold Mₕ can be set appropriately.

f) The first somatosensory information may be fat information of the first limb, e.g., first fat information. The step S140 may be:

S140f: determining whether the first limb is a dominant limb according to the first fat information and reference information.

The first fat information may be fat ratio information acquired on the first limb. The first fat information may be fat ratio information of a corresponding part of the body of the user in a period of time, and may be acquired by using at least one fat sensor that is close to or in contact with the skin of the user.

The inventor has found that as a natural result of evolution, the fat content of a dominant limb of a person is generally higher than the fat content of a non-dominant limb. A high fat content can protect muscles and artery vessels of the dominant limb that performs more actions from injuries, and also provides more energy.

In a currently mainstream method, the fat content in the human body, e.g., the fat ratio, is obtained by measuring body resistance, which is based on such a principle that muscles contain more fluids such as blood and presents a low resistance while fat presents a high resistance. Therefore, the resistance can be calculated by introducing a small electrical current to pass through the body, and then the fat ratio can be measured according to the resistance.

FIG. 17 is a schematic diagram of comparison between fat information of a dominant hand and a non-dominant hand of a user, where the abscissa represents time, the ordinate represents a fat ratio, a solid curve represents a fat ratio curve of the dominant hand, and a dashed curve represents a fat ratio curve of the non-dominant hand. It can be seen that, the fat ratio of the dominant hand is generally higher than the fat ratio of the non-dominant hand.

The inventor has also found that a difference similar to that shown in FIG. 17 also exists between fat information of other limbs (for example, arm, foot or leg) of the user. Determination of a dominant limb can be implemented based on the above principle.

In one implementation manner, the reference information is second fat information of a second limb of the user. The method may further comprise:

S130f: acquiring the second fat information of the second limb of the user as the reference information.

For example, two sets of fat sensors may be disposed, to collect fat information of the first limb and the second limb of the user at the same time, and the fat information collected on the second limb, e.g., the second fat information, is used as the reference information.

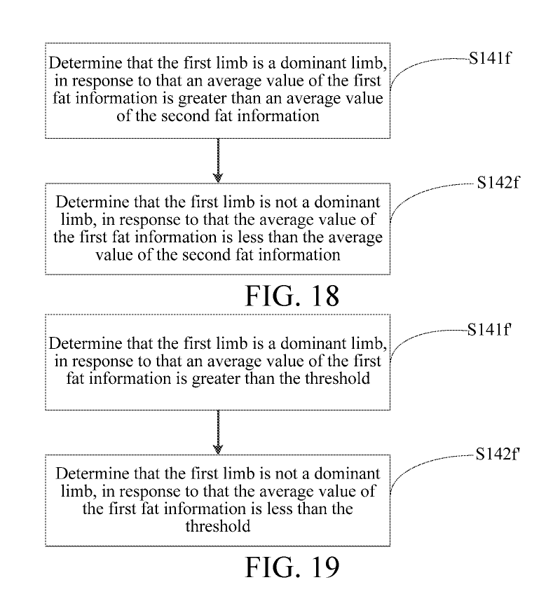

In this implementation manner, in the step S140f, it may be determined whether the first limb is a dominant limb by comparing an average value of the first fat information with an average value of the second fat information. The average value of the first fat information is an average value of fat ratios corresponding to multiple sampling time points in the first fat information, and similarly, the average value of the second fat information is an average value of fat ratios corresponding to multiple sampling time points in the second fat information. The use of the average value can avoid the problem that a sampling error occurring at a single sampling time point leads to incorrect determination of a dominant limb, thereby improving the accuracy of dominant limb determination. Specifically, as shown in FIG. 18, the step S140f may comprise:

S141f: determining that the first limb is a dominant limb, in response to that an average value of the first fat information is greater than an average value of the second fat information; and

S142f: determining that the first limb is not a dominant limb, in response to that the average value of the first fat information is less than the average value of the second fat information.

In another implementation manner, the reference information may be a threshold determined according to an average value of left-limb fat information and an average value of right-limb fat information of the user. Specifically, as shown in FIG. 19, the step S140 may comprise:

S141f: determining that the first limb is a dominant limb, in response to that an average value of the first fat information is greater than the threshold; and\

S142f: determining that the first limb is not a dominant limb, in response to that the average value of the first fat information is less than the threshold.

For example, the right-limb fat information and the left-limb fat information of the user are collected in advance, analyzed and processed. Assuming that the average value of the right-limb fat information falls within a first range (R_{fmin}, R_{fmax}), assuming that the average value of the left-limb fat information falls within a second range (L_{fmin}, L_{fmax}), and assuming that the right limb is a dominant limb, it is obtained that L_{fmax}<R_{fmin}, and it can be determined that the threshold is M_{f}, and L_{fmax}<M_{f}<R_{fmin}. That is, the threshold M_{f} is a value between the first range and the second range.

Therefore, if the average value of the first fat information is greater than the threshold M_{f}, it is considered that it falls within the first range, and the first limb is a dominant limb of the user; if the average value of the first fat information is less than the threshold M_{f}, it is considered that it falls within the second range, and the first limb is not a dominant limb of the user.

Generally, an average value of fat information of a dominant limb is at least 0.7% higher than an average value of fat information of a non-dominant limb, based on which the threshold M_{f} can be set appropriately.

g) The first somatosensory information may be alkalinity/acidity information of the first limb, e.g., first alkalinity/acidity information. The step S140 may be:

S140g: determining whether the first limb is a dominant limb according to the first alkalinity/acidity information and reference information.

The first alkalinity/acidity information may be alkalinity/acidity information acquired on the first limb. The first alkalinity/acidity information may be PH value information of a corresponding part of the body of the user in a period of time, and may be acquired by using at least one alkalinity/acidity sensor that is close to or in contact with the skin of the user.

The inventor has found that because a dominant limb is rougher than a non-dominant limb and the skin layer of the dominant limb is thicker than the skin layer of the non-dominant limb, the alkalinity/acidity (e.g., PH value) of the dominant limb of the user is generally higher than the alkalinity/acidity of the non-dominant limb.

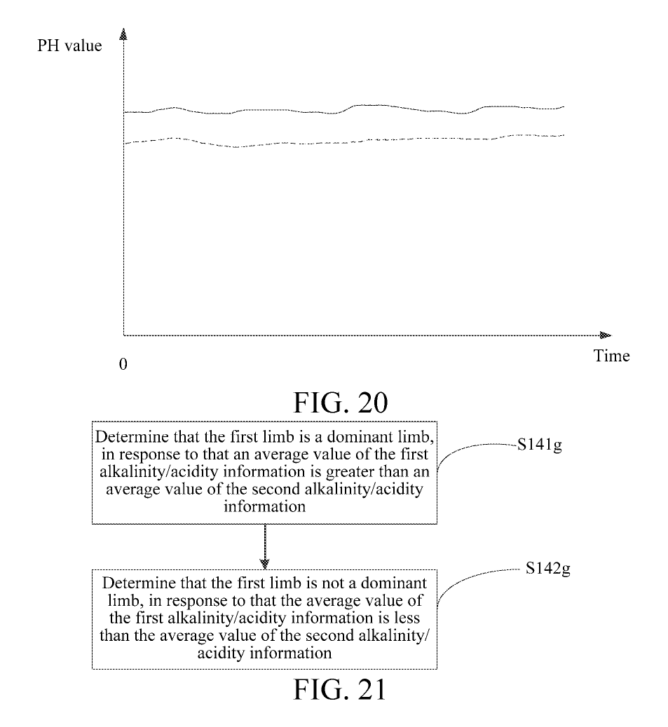

FIG. 20 is a schematic diagram of comparison between alkalinity/acidity information of a dominant hand and a non-dominant hand of a user, where the abscissa represents time, the ordinate represents a PH value, a solid curve represents an alkalinity/acidity curve of the dominant hand, and a dashed curve represents an alkalinity/acidity curve of the non-dominant hand. It can be seen that, the alkalinity/acidity of the dominant hand is generally higher than the alkalinity/acidity of the non-dominant hand.

The inventor has also found that a difference similar to that shown in FIG. 20 also exists between alkalinity/acidity information of other limbs (for example, arm, foot or leg) of the user. Determination of a dominant limb can be implemented based on the above principle.

In one implementation manner, the reference information is second alkalinity/acidity information of a second limb of the user. The method may further comprise:

S130g: acquiring the second alkalinity/acidity information of the second limb of the user as the reference information.

For example, two sets of alkalinity/acidity sensors may be disposed, to collect alkalinity/acidity information of the first limb and the second limb of the user at the same time, and the alkalinity/acidity information collected on the second limb, e.g., the second alkalinity/acidity information, is used as the reference information.

In this implementation manner, in the step S140g, it may be determined whether the first limb is a dominant limb by comparing an average value of the first alkalinity/acidity information with an average value of the second alkalinity/acidity information. The average value of the first alkalinity/acidity information is an average value of PH values corresponding to multiple sampling time points in the first alkalinity/acidity information, and similarly, the average value of the second alkalinity/acidity information is an average value of PH values corresponding to multiple sampling time points in the second alkalinity/acidity information. The use of the average value can avoid the problem that a sampling error occurring at a single sampling time point leads to incorrect determination of a dominant limb, thereby improving the accuracy of dominant limb determination. Specifically, as shown in FIG. 21, the step S140g may comprise:

S141g: determining that the first limb is a dominant limb, in response to that an average value of the first alkalinity/acidity information is greater than an average value of the second alkalinity/acidity information; and

S142g: determining that the first limb is not a dominant limb, in response to that the average value of the first alkalinity/acidity information is less than the average value of the second alkalinity/acidity information.

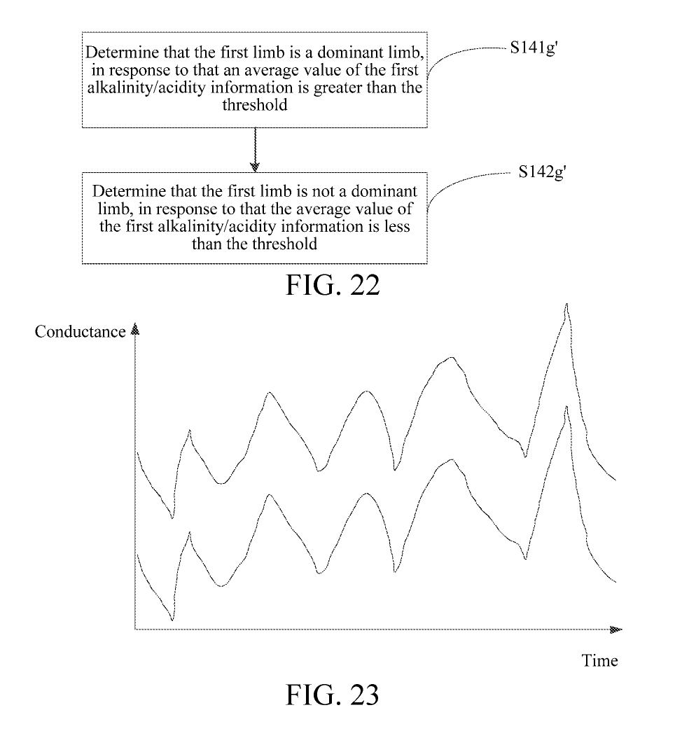

In another implementation manner, the reference information may be a threshold determined according to an average value of left-limb alkalinity/acidity information and an average value of right-limb alkalinity/acidity information of the user. Specifically, as shown in FIG. 22, the step S140g may comprise:

S141g′: determining that the first limb is a dominant limb, in response to that an average value of the first alkalinity/acidity information is greater than the threshold; and

S142g′: determining that the first limb is not a dominant limb, in response to that the average value of the first alkalinity/acidity information is less than the threshold.

For example, the right-limb alkalinity/acidity information and the left-limb alkalinity/acidity information of the user are collected in advance, analyzed and processed. Assuming that the average value of the right-limb alkalinity/acidity information falls within a first range (Rₚₘᵢₙ, Rₚₘₐₓ), assuming that the average value of the left-limb alkalinity/acidity information falls within a second range (Lₚₘᵢₙ, Lₚₘₐₓ), and assuming that the right limb is a dominant limb, it is obtained that Lₚₘₐₓ<Rₚₘᵢₙ, and it can be determined that the threshold is Mₚ, and Lₚₘₐₓ<Mₚ<Rₚₘᵢₙ. That is, the threshold Mₚ is a value between the first range and the second range.

Therefore, if the average value of the first alkalinity/acidity information is greater than the threshold Mₚ, it is considered that it falls within the first range, and the first limb is a dominant limb of the user; if the average value of the first alkalinity/acidity information is less than the threshold Mₚ, it is considered that it falls within the second range, and the first limb is not a dominant limb of the user.

Generally, an average value of alkalinity/acidity information of a dominant limb is at least 0.15 higher than an average value of alkalinity/acidity information of a non-dominant limb, based on which the threshold M_{f} can be set appropriately.

h) The first somatosensory information may be skin conductance information of the first limb, e.g., first skin conductance information. The step S140 may be:

S140h: determining (or identifying) whether the limb is a dominant limb according to the first skin conductance information and reference information.

The first skin conductance information may be eigenvalues of the skin conductance level (skin conductance level), and may be acquired by using a skin conductance sensor that is in contact with the skin of the user. Generally all existing smart wristbands, smart watches and the like are equipped with the sensor; therefore, the implementation of the method does not increase the hardware costs of the existing wearable device.

The inventor has found that according to the principle of skin conductance, the skin conductance of the dominant hand of the user is statistically significantly different from the skin conductance of the non-dominant hand, e.g., a variance of skin conductance signals of the dominant hand and skin conductance signals of the non-dominant hand is less than or equal to 0.05. As shown in FIG. 23, an upper curve represents a curve of skin conductance information obtained by sampling skin conductance signals of a non-dominant hand of a user, a lower curve represents a curve of skin conductance information obtained by sampling skin conductance signals of a dominant hand of the user. The transverse axis represents time in seconds, the longitudinal axis represents conductance in microsiemens. It can be seen that the two curves are generally different, and it can be obtained through analysis that the average amplitude value of the skin conductance information of the dominant hand is generally less than the average amplitude value of the skin conductance information of the non-dominant hand. The amplitude in the present application is an amplitude of a waveform corresponding to the skin conductance signal, and its value is non-negative.

The inventor has also found that a difference similar to that shown in FIG. 23 also exists between skin conductance information of other limbs (for example, arm, foot or leg) of the user. Determination of a dominant limb can be implemented based on the above principle.

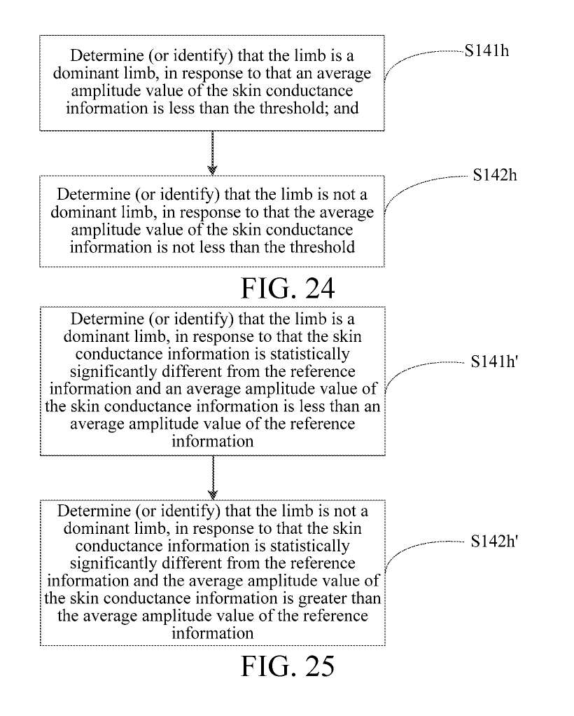

In one implementation manner, the reference information is a threshold determined according to left-limb skin conductance information and right-limb skin conductance information of the user, and in the step S140h, it may be determined (or identified) whether the limb is a dominant limb according to the skin conductance information and the threshold; and referring to FIG. 24, the step S140h may further comprise:

S141h: determining (or identifying) that the limb is a dominant limb, in response to that an average amplitude value of the skin conductance information is less than the threshold; and

S142h: determining (or identifying) that the limb is not a dominant limb, in response to that the average amplitude value of the skin conductance information is not less than the threshold.

For example, the reference information may be a threshold determined according to left-hand skin conductance information and right-hand skin conductance information of the user. Assuming that an average amplitude value of the left-hand skin conductance information of the user falls within a first range (Lₘᵢₙ, Lₘₐₓ), assuming that an average amplitude value of the right-hand skin conductance information of the user falls within a second range (Rₘᵢₙ, Rₘₐₓ), and assuming that the left hand of the user is a dominant hand, it is obtained that Lₘₐₓ<Rₘᵢₙ, and it can be determined that the threshold is M, and Lₘₐₓ<M<Rₘᵢₙ. That is, the threshold M is a value between the first range and the second range.

Therefore, if the average amplitude value of the skin conductance information is less than the threshold M, it is considered that it falls within the first range, and the limb is a dominant hand; if the average amplitude value of the skin conductance information is not less than the threshold M, it is considered that it falls within the second range, and the limb is not a dominant hand.

It should be noted that, in this implementation manner, the reference information needs to be determined according to the left-limb skin conductance information and the right-limb skin conductance information of the user; therefore, the left-limb skin conductance information and the right-limb skin conductance information of the user need to be acquired in advance, for example, before formally using the dominant limb determination (or identification) device, the user wears the device on the left hand for a period of time and then wears the device on the right hand for a period of time, so as to complete a training process.

In another implementation manner, the reference information is left-limb skin conductance information or right-limb skin conductance information of the user, and in the step S140h, it may be determined (or identified) whether the limb is a dominant limb by comparing the skin conductance information with the reference information; and referring to FIG. 25, the step S140h may further comprise:

S141h′: determining (or identifying) that the limb is a dominant limb, in response to that the skin conductance information is statistically significantly different from the reference information and an average amplitude value of the skin conductance information is less than an average amplitude value of the reference information.

The step S140h may further comprise:

S142h′: determining (or identifying) that the limb is not a dominant limb, in response to that the skin conductance information is statistically significantly different from the reference information and the average amplitude value of the skin conductance information is greater than the average amplitude value of the reference information.

In this implementation manner, the user does not need to perform training in advance, and for example, in response to that the left hand or the right hand of the user wears the dominant limb determination (or identification) device for the first time, the first skin conductance information is acquired, which is used as the reference information; in response to that the user wears the dominant limb determination (or identification) device again after a period of time (for example, on the next day), the second skin conductance information is acquired. The first skin conductance information and the second skin conductance information may be skin conductance information of a same hand or may respectively be skin conductance information of two hands, and according to the above principle for determining (or identifying) a dominant hand, the dominant hand of the user can be determined (or identified) only when the first skin conductance information and the second skin conductance information are respectively skin conductance information of two hands.

Therefore, in the steps S141h′ and S142h′, the condition that the skin conductance information is statistically significantly different from the reference information needs to be satisfied first, indicating that the skin conductance information and the reference information are respectively skin conductance information of two hands, and then the average amplitude value of the skin conductance information may be compared with the average amplitude value of the reference information, so as to complete the determination (or identification) process.

This implementation manner is advantageous in that the user does not need to complete a training process intentionally, and collection of the reference information is completed during normal use by the user; however, the method in this implementation manner still requires acquiring skin conductance information of both two hands in turn, and is mainly applicable to scenarios in which the user wears a wearable device on two hands alternately.

In addition, it should be understood by a person skilled in the art that when a user has worn the dominant limb determination (or identification) device on two hands, skin conductance information of one hand can be properly selected as the reference information by using the method, to ensure that the skin conductance information is statistically significantly different from the reference information, so as to complete the determination (or identification) process.

In another implementation manner, the reference information is second skin conductance information of a second limb of the user. The method may further comprise:

S130h: acquiring the second skin conductance information of the second limb of the user as the reference information.

For example, two sets of skin conductance sensors may be disposed, to collect skin conductance information of the first limb and the second limb of the user at the same time, and the skin conductance information collected on the second limb, e.g., the second skin conductance information, is used as the reference information.

In this implementation manner, in the step S140, it may be determined whether the first limb is a dominant limb by comparing an average amplitude value of the first skin conductance information with an average amplitude value of the second skin conductance information. The average amplitude value of the first skin conductance information is an average amplitude value of skin conductance amplitude values corresponding to multiple sampling time points in the first skin conductance information, and similarly, the average amplitude value of the second skin conductance information is an average amplitude value of skin conductance amplitude values corresponding to multiple sampling time points in the second skin conductance information.

The use of the average amplitude value in the present application can avoid the problem that a sampling error occurring at a single sampling time point leads to incorrect determination of a dominant limb, thereby improving the accuracy of dominant limb determination.

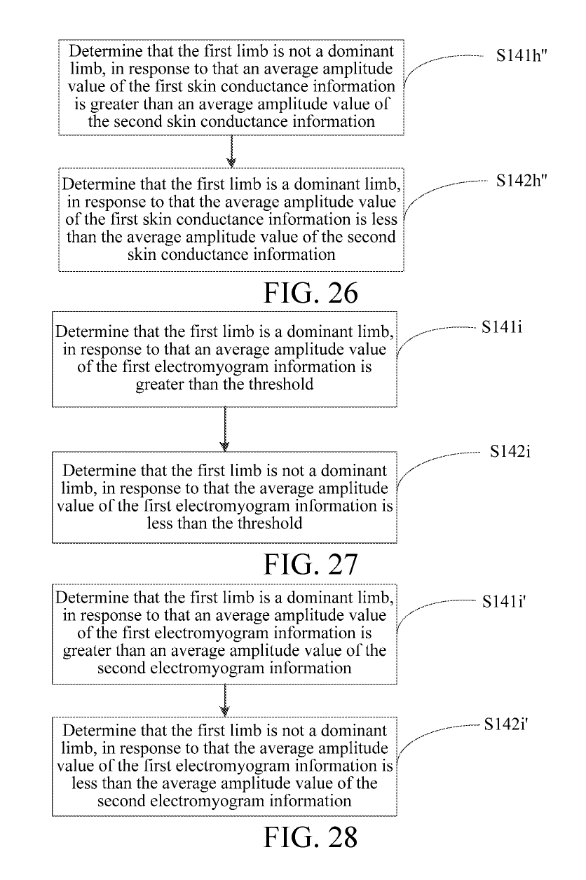

Specifically, and referring to FIG. 26, the step S140 may comprise:

S141h″: determining that the first limb is not a dominant limb, in response to that an average amplitude value of the first skin conductance information is greater than an average amplitude value of the second skin conductance information; and

S142h″: determining that the first limb is a dominant limb, in response to that the average amplitude value of the first skin conductance information is less than the average amplitude value of the second skin conductance information.

i) The first somatosensory information may be electromyogram information of the first limb, e.g., first electromyogram information. The step S140 may be:

S140i: determining (or identifying) whether the first limb is a dominant limb according to the first electromyogram information and reference information.

The first electromyogram information may be electromyogram signals in a period of time, and may be acquired by using a set of electromyogram sensors that is in contact with the skin of the user. The set of electromyogram sensors comprises one or more electromyogram sensors.

The inventor has found that when muscles contract at different loads, the amplitude value of the electromyogram information is proportional to the muscle strength, e.g., the larger the tension generated by muscles is, the larger the amplitude value of the electromyogram information is. Further, when muscles contract at 40% MVC (maximum voluntary contraction), the muscle strength is in linear relationship with the electromyogram amplitude value; when muscles contract at 60% MVC or higher, the muscle strength is also in linear relationship with the electromyogram amplitude value, but the straight slope is greater in this case. When the muscle strength is 40%-60% MVC, the muscle strength is not in linear relationship with, but is still proportional to, the amplitude value of the electromyogram information.

In addition, the inventor has also found that a dominant limb of a person may be used more frequently than a non-dominant limb, in other words, the frequency of muscle contraction of a dominant limb of a person is generally higher than that of a non-dominant limb. Therefore, the inventor has found during research that after electromyogram information of a same muscle or muscle group of a dominant limb and a non-dominant limb of a user is sampled at a same sampling frequency within a period of time (for example, one hour), an average amplitude value of the electromyogram information of the dominant limb is generally higher than an average amplitude value of the electromyogram information of the non-dominant limb. In the present application, dominant limb determination (identification) is implemented based on the above principle.

In one implementation manner, the reference information is a threshold determined according to an average amplitude value of left-limb electromyogram information and an average amplitude value of right-limb electromyogram information of the user.

Referring to FIG. 27, the step S140i may comprise:

S141i: determining that the first limb is a dominant limb, in response to that an average amplitude value of the first electromyogram information is greater than the threshold; and

S142i: determining that the first limb is not a dominant limb, in response to that the average amplitude value of the first electromyogram information is less than the threshold.

For example, the reference information may be a threshold determined according to an average amplitude value of left-hand electromyogram information and an average amplitude value of right-hand electromyogram information of the user. Assuming that the average amplitude value of the left-hand electromyogram information of the user falls within a first range (Lₘᵢₙ, Lₘₐₓ), assuming that the average amplitude value of the right-hand electromyogram information of the user falls within a second range Rₘᵢₙ, Rₘₐₓ), and assuming that the left hand of the user is a dominant hand, it is obtained that Lₘᵢₙ>Rₘₐₓ, and it can be determined that the threshold is M, and Lₘᵢₙ>M>Rₘₐₓ. That is, the threshold M is a value between the first range and the second range.

Therefore, if the average value of the electromyogram information is greater than the threshold M, it is considered that it falls within the first range, and the limb is a dominant hand; if the average value of the electromyogram information is less than the threshold M, it is considered that it falls within the second range, and the limb is not a dominant hand.

It should be noted that, in this implementation manner, the reference information needs to be determined according to the left-limb electromyogram information and the right-limb electromyogram information of the user; therefore, the left-limb electromyogram information and the right-limb electromyogram information of the user need to be acquired in advance, for example, before formally using the dominant limb determination (or identification) device, the user wears the device on the left hand for a period of time and then wears the device on the right hand for a period of time, so as to complete a training process.

In another implementation manner, the reference information is second electromyogram information of a second limb of the user. The method may further comprise:

S130: acquiring the second electromyogram information of the second limb of the user as the reference information. Referring to FIG. 28, the step S140i may comprise:

S141i′: determining that the first limb is a dominant limb, in response to that an average amplitude value of the first electromyogram information is greater than an average amplitude value of the second electromyogram information; and

S142i′: determining that the first limb is not a dominant limb, in response to that the average amplitude value of the first electromyogram information is less than the average amplitude value of the second electromyogram information.

In this implementation manner, the user does not need to perform training in advance, and for example, may separately acquire the first electromyogram information of the first limb and the second electromyogram information of the second limb of the user, and then compare the average amplitude value of the first electromyogram information with the average amplitude value of the second electromyogram information, so as to determine (or identify) a dominant limb.

This implementation manner is advantageous in that the user does not need to complete a training process intentionally, and collection of the reference information is completed during normal use by the user; however, the method in this implementation manner may require acquiring electromyogram information of, for example, both two hands, and is mainly applicable to scenarios in which the user needs to use the two hands at the same time, for example, the user wears smart gloves on the two hands to play an electronic game.

j) The first somatosensory information may be bio-impedance information of the first limb, e.g., first bio-impedance information. The step S140 may be:

S140j: determining whether the first limb is a dominant limb according to the first bio-impedance information and reference information.

The bio-impedance is one of the most important physiological indicators of the human body. In a bio-impedance technology, biomedical information related to physiological and pathological conditions of the human body is extracted by using impedance characteristics of biological tissues and organs and variations thereof. An electrode system arranged on the body surface is used to supply a small alternating current measuring current to an object to be detected, detect a corresponding electrical impedance and its changes, and then acquire relevant physiological and pathological information in such aspects as cell measurement, volume measurement, analysis of the structure of the human body and analysis of the composition of the human body according to different purposes of application.

By taking measurement of a bio-impedance at an arm of the human body, the current flows through the following path: an electrode plate on one side, skin on one side, various tissues under the skin, skin on the other side, and an electrode plate on the other side. The bio-impedance of the human body is the sum of the impedance of the skin and the impedances of various tissues under the skin. The epidermis is the outer layer of the skin and has poor electrical conduction. There are various substances having different resistivity under the skin, which are formed by various cells.

The first bio-impedance information may be bio-impedance information acquired on the first limb. The first bio-impedance information may be bio-impedance signals in a period of time, and may be acquired by using at least one bio-impedance sensor that is close to or in contact with the skin of the user.

The inventor has found that because a dominant limb of a person is generally larger and stronger than a non-dominant limb and has rougher skin and thicker skin layer than the non-dominant limb, the bio-impedance of the dominant limb is generally higher than the bio-impedance of the non-dominant limb.

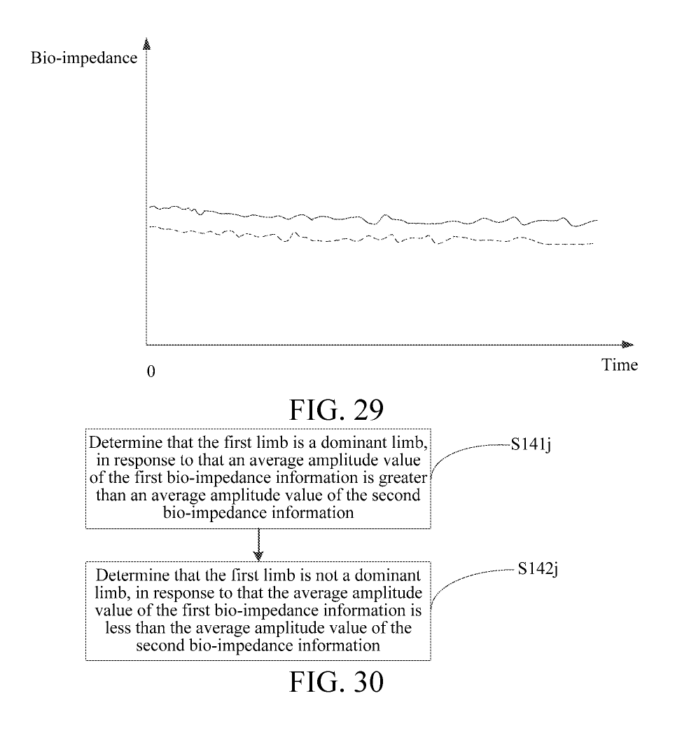

FIG. 29 is a schematic diagram of comparison between bio-impedance signals of a dominant hand and a non-dominant hand of a user, where the abscissa represents time, the ordinate represents a bio-impedance amplitude value in ohms, a solid curve represents a bio-impedance curve of the dominant hand, and a dashed curve represents a bio-impedance curve of the non-dominant hand. It can be seen that, the bio-impedance amplitude value of the dominant hand is generally higher than the bio-impedance amplitude value of the non-dominant hand. The amplitude in the present application is an amplitude of a waveform corresponding to the bio-impedance signal, and its value is non-negative.

The inventor has also found that a difference similar to that shown in FIG. 23 also exists between bio-impedance signals of other limbs (for example, arm, foot or leg) of the user. Determination of a dominant limb can be implemented based on the above principle.

In one implementation manner, the reference information is second bio-impedance information of a second limb of the user. The method may further comprise:

S130j: acquiring the second bio-impedance information of the second limb of the user as the reference information.

For example, two sets of bio-impedance sensors may be disposed, to collect bio-impedance information of the first limb and the second limb of the user at the same time, and the bio-impedance information collected on the second limb, e.g., the second bio-impedance information, is used as the reference information.

In this implementation manner, in the step S140j, it may be determined whether the first limb is a dominant limb by comparing an average amplitude value of the first bio-impedance information with an average amplitude value of the second bio-impedance information. The average amplitude value of the first bio-impedance information is an average value of bio-impedance amplitude values corresponding to multiple sampling time points in the first bio-impedance information, and similarly, the average amplitude value of the second bio-impedance information is an average value of bio-impedance amplitude values corresponding to multiple sampling time points in the second bio-impedance information. The use of the average amplitude value can avoid the problem that a sampling error occurring at a single sampling time point leads to incorrect determination of a dominant limb, thereby improving the accuracy of dominant limb determination. Specifically, as shown in FIG. 30, the step S140j may comprise:

S141j: determining that the first limb is a dominant limb, in response to that an average amplitude value of the first bio-impedance information is greater than an average amplitude value of the second bio-impedance information; and

S142j: determining that the first limb is not a dominant limb, in response to that the average amplitude value of the first bio-impedance information is less than the average amplitude value of the second bio-impedance information.

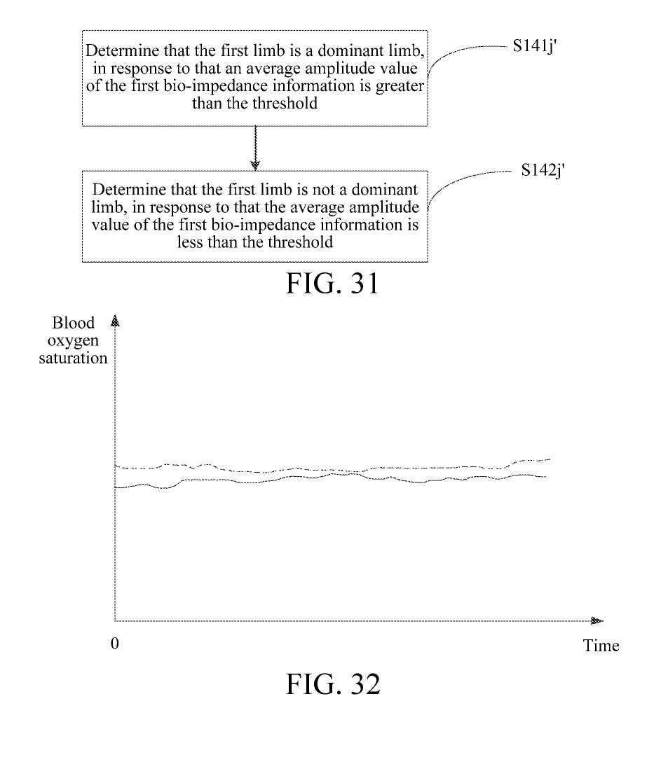

In another implementation manner, the reference information may be a threshold determined according to an average amplitude value of left-limb bio-impedance information and an average amplitude value of right-limb bio-impedance information of the user. Specifically, as shown in FIG. 31, the step S140j may comprise:

S141j′: determining that the first limb is a dominant limb, in response to that an average amplitude value of the first bio-impedance information is greater than the threshold; and

S142j′: determining that the first limb is not a dominant limb, in response to that the average amplitude value of the first bio-impedance information is less than the threshold.

For example, the right-limb bio-impedance information and the left-limb bio-impedance information of the user are collected in advance, analyzed and processed. Assuming that the average amplitude value of the right-limb bio-impedance information falls within a first range (Rᵣₘᵢₙ, Rᵣₘₐₓ), assuming that the average amplitude value of the left-limb bio-impedance information falls within a second range (Lᵣₘᵢₙ, Lᵣₘₐₓ), and assuming that the right limb is a dominant limb, it is obtained that Lᵣₘₐₓ<Rᵣₘᵢₙ, and it can be determined that the threshold is Mᵣ, and Lᵣₘₐₓ<Mᵣ<Rᵣₘᵢₙ. That is, the threshold Mᵣ is a value between the first range and the second range.

Therefore, if the average amplitude value of the first bio-impedance information is greater than the threshold Mᵣ, it is considered that it falls within the first range, and the first limb is a dominant limb of the user; if the average amplitude value of the first bio-impedance information is less than the threshold Mᵣ, it is considered that it falls within the second range, and the first limb is not a dominant limb of the user.

Generally, an average amplitude value of bio-impedance information of a dominant limb is at least 5% higher than an amplitude average value of bio-impedance information of a non-dominant limb, based on which the threshold Mᵣ can be set appropriately.

k) The first somatosensory information may be blood oxygen information of the first limb, e.g., first blood oxygen information. The step S140 may be:

S140k: determining whether the first limb is a dominant limb according to the first blood oxygen information and reference information.

The blood oxygen information may be blood oxygen saturation information in a period of time, and may be acquired by using at least one blood oxygen sensor that is close to or in contact with the skin of the user. The blood oxygen saturation (SaO2) is the percentage of oxyhemoglobin (HbO2) in all hemoglobin that can be oxygenated in the blood, e.g., the blood oxygen concentration in the blood, and is an important physiological parameter of respiratory circulation. The blood oxygen saturation may be acquired by means of reflection or transmission. The first blood oxygen information is blood oxygen information acquired on the first limb.

The inventor has found that the metabolic process of the human body is a biological oxidation process, and oxygen needed in the metabolic process enters the blood in the human body through the respiratory system, is bound to hemoglobin in red blood cells in the blood to form oxyhemoglobin, and is then conveyed to histocytes of various parts of the human body. Because the dominant limb of the user generally moves more frequently than the non-dominant limb and consumes more oxygen, an average value of the blood oxygen saturation of the dominant limb of the user may be lower than an average value of the blood oxygen saturation of the non-dominant hand.

FIG. 32 is a schematic diagram of comparison between blood oxygen information of a dominant hand and a non-dominant hand of a user, where the abscissa represents time, the ordinate represents a blood oxygen saturation, a solid curve represents a blood oxygen information curve of the dominant hand, and a dashed curve represents a blood oxygen information curve of the non-dominant hand. It can be seen that, the blood oxygen saturation of the non-dominant hand is generally higher than the blood oxygen saturation of the dominant hand.

The inventor has also found that a difference similar to that shown in FIG. 32 also exists between blood oxygen information of other limbs (for example, arm, foot or leg) of the user. Determination of a dominant limb can be implemented based on the above principle.

In one implementation manner, the reference information is second blood oxygen information of a second limb of the user. The method may further comprise:

S130k: acquiring the second blood oxygen information of the second limb of the user as the reference information.

For example, two sets of blood oxygen sensors may be disposed, to collect blood oxygen information of the first limb and the second limb of the user at the same time, and the blood oxygen information collected on the second limb, e.g., the second blood oxygen information, is used as the reference information.

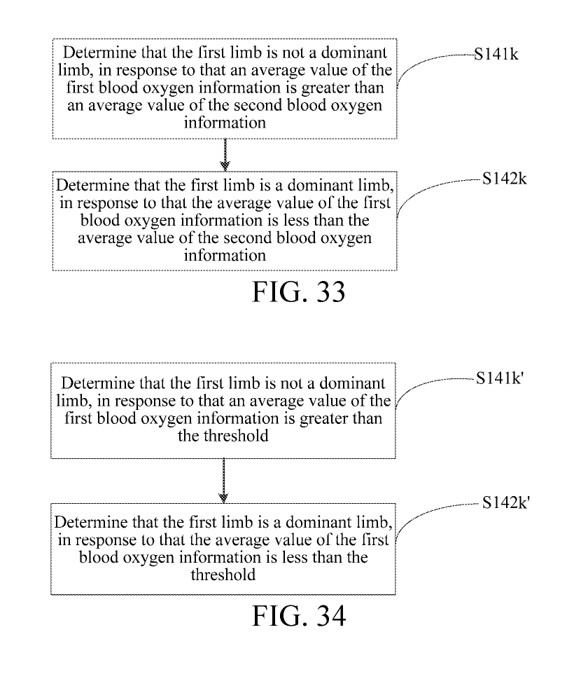

In this implementation manner, in the step S140k, it may be determined whether the first limb is a dominant limb by comparing an average value of the first blood oxygen information with an average value of the second blood oxygen information. The average value of the first blood oxygen information is an average value of blood oxygen saturations corresponding to multiple sampling points in the first blood oxygen information, and similarly, the average value of the second blood oxygen information is an average value of blood oxygen saturations corresponding to multiple sampling points in the second blood oxygen information. The use of the average value can avoid the problem that a sampling error occurring at a single sampling time point leads to incorrect determination of a dominant limb, thereby improving the accuracy of dominant limb determination. Specifically, as shown in FIG. 33, the step S140k may comprise:

S141k: determining that the first limb is not a dominant limb, in response to that an average value of the first blood oxygen information is greater than an average value of the second blood oxygen information; and

S142k: determining that the first limb is a dominant limb, in response to that the average value of the first blood oxygen information is less than the average value of the second blood oxygen information.

In another implementation manner, the reference information may be a threshold determined according to an average value of left-limb blood oxygen information and an average value of right-limb blood oxygen information of the user. Specifically, as shown in FIG. 34, the step S140k may comprise:

S141k′: determining that the first limb is not a dominant limb, in response to that an average value of the first blood oxygen information is greater than the threshold; and

S142k′: determining that the first limb is a dominant limb, in response to that the average value of the first blood oxygen information is less than the threshold.

For example, the right-limb blood oxygen information and the left-limb blood oxygen information of the user are collected in advance, analyzed and processed. Assuming that the average value of the right-limb blood oxygen information falls within a first range Rₒₘᵢₙ, Rₒₘₐₓ), assuming that the average value of the left-limb blood oxygen information falls within a second range (Lₒₘᵢₙ, Lₒₘₐₓ), and assuming that the left limb is a dominant limb, it is obtained that Lₒₘₐₓ<Rₒₘᵢₙ, and it can be determined that the threshold is Mₒ, and Lₒₘₐₓ<Mₒ<Rₒₘᵢₙ. That is, the threshold Mₒ is a value between the first range and the second range.

Therefore, if the average value of the first blood oxygen information is greater than the threshold Mₒ, it is considered that it falls within the first range, and the first limb is not a dominant limb of the user; if the average value of the first blood oxygen information is less than the threshold Mₒ, it is considered that it falls within the second range, and the first limb is a dominant limb of the user.

Generally, an average value of blood oxygen information of a non-dominant limb is at least 1% higher than an average value of blood oxygen information of a dominant limb, based on which the threshold Mₒ can be set appropriately.

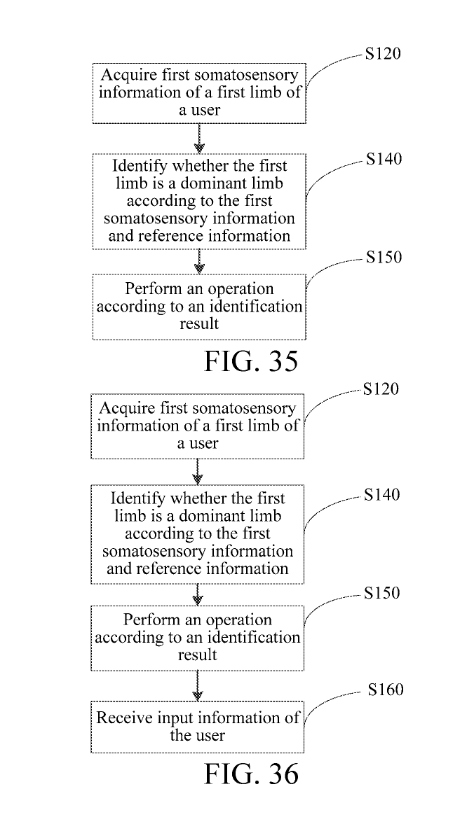

Referring to FIG. 35, in one implementation manner, the method further comprises:

S150: performing an operation according to an identification result.

For example, it is learned according to the identification result that the smart wristband is worn on the dominant hand of the user, and since the dominant hand is often needed during work, a prompt may be provided when the arm of the dominant hand of the user moves frequently, to remind the user to take care of the wristband to avoid collision.

For another example, the user wears smart gloves on two hands to play an electronic shooting game, and if the gloves identify that the left hand of the user is the dominant hand, the gloves can automatically switch to a right-hand gun holding mode, so as to better adapt to use habits of the user.

In addition, according to the identification result and input information of the user, it may further be identified whether a hand is the left or right hand of the user or whether the user is left-handed.

Referring to FIG. 36, in one implementation manner, the method further comprises:

S160: receiving input information of the user.

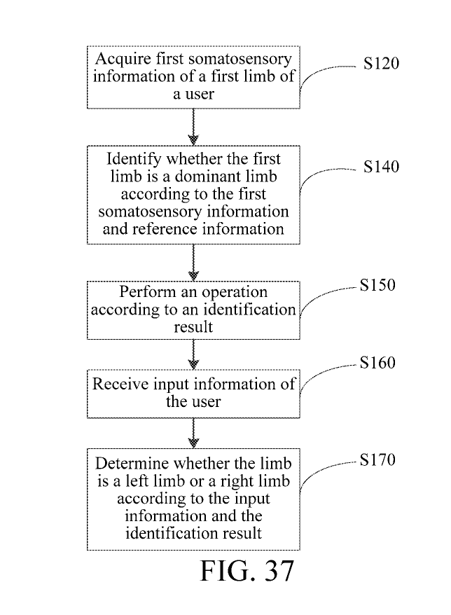

Referring to FIG. 37, in one implementation manner, the input information is dominant-limb information, e.g., the user is left-handed or right-handed. In this implementation manner, the method further comprises:

S170: determining whether the limb is a left limb or a right limb according to the input information and the identification result.

For example, if the dominant-limb information indicates that the user is left-handed, and the identification result indicates that the first limb is a dominant hand, it may be determined that the first limb is the left hand.

After it is determined that the first limb is the left limb or the right limb, settings such as a display interface of the wearable device worn on the first limb of the user can be adaptively adjusted, or settings such as a display interface of a device such as a smart phone held by the first limb may further be adjusted.

For example, in the method, assuming that it is determined that the first limb is the left hand of the user, an unlock gesture for a smart watch worn on the first limb may be set to be sliding from left to right, and a smart phone may be set to display consonants on the left half of the screen and display vowels on the right half of the screen.

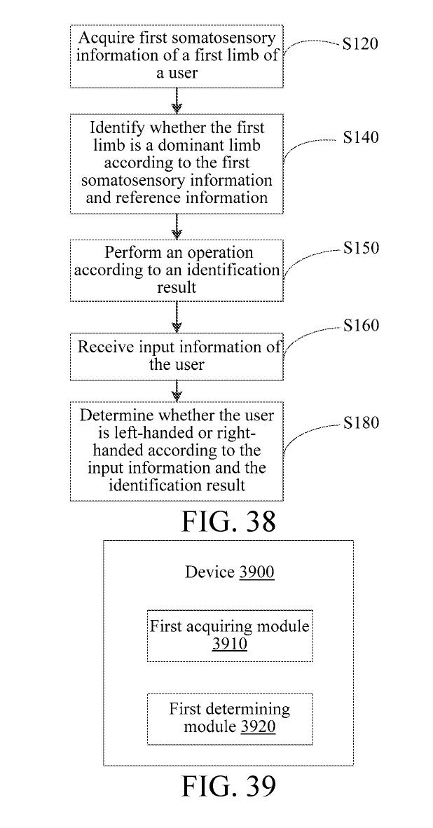

Referring to FIG. 38, in one implementation manner, the input information comprises that the first limb is the left hand or the right hand, and the method may further comprise:

S180: determining whether the user is left-handed or right-handed according to the input information and an identification result.

For example, the input information shows that the first limb is the left hand, and the identification result indicates that the first limb is a dominant hand, it may be determined that the user is left-handed. The input information may be a voice input, a text input, or the like, or may be a motion input. For example, when the user wears a left-hand glove on one hand, it is equivalent to inputting that the one hand is the left hand.

In addition, an embodiment of the present application further provides a computer-readable medium, comprising computer-readable instructions that when executed, perform the following operations: performing the operations of the steps S120 and S140 of the method in the foregoing implementation manner shown in FIG. 1.

Based on the above, in the method according to this embodiment of the present application, it can be identified according to first somatosensory information of a first limb of a user and reference information whether the first limb is a dominant limb; further, it can be determined according to input information of the user whether the limb is a left limb or a right limb, or whether the user is left-handed; and a corresponding operation is performed according to the identification result or determining result, which reduces the number of setting operations that needs to be performed by the user, thereby improving user experience.

FIG. 39 is a schematic structural block diagram of a dominant limb identification device according to one embodiment of the present application. The dominant limb identification device may be disposed as one functional module in a wearable device such as a smart wristband, a smart watch or a smart glove, and definitely may serve as an independent wearable device for used by a user. As shown in FIG. 39, the device 3900 may comprise a first acquiring module 3910, configured to acquire first somatosensory information of a first limb of a user and a first determining module 3920, configured to determine (or identify) whether the first limb is a dominant limb according to the first somatosensory information and reference information.

The device according to this embodiment of the present application acquires first somatosensory information of a first limb of a user, and identifies whether the first limb is a dominant limb according to the first somatosensory information and reference information. In this way, a device for determining a dominant limb is provided, which enables a device worn by the user to perform automatic setting according to the identification result, thereby improving user experience.

The functions of the first acquiring module 3910 and the first determining module 3920 will be described in detail below with reference to specific implementation manners.

The first acquiring module 3910 is configured to acquire first somatosensory information of a first limb of a user.

The first limb may be one of the two hands of the user or one of the two arms of the user. For brevity, an example in which the first limb is one of the two hands of the user is mainly described below.

The first somatosensory information may be acceleration information, blood flow information, PPG information, temperature information, humidity information, fat information, alkalinity/acidity information, skin conductance information, electromyogram information, bio-impedance information or blood oxygen information of the first limb, which may be collected/acquired by using a corresponding sensor. For example, the acceleration information of the first limb may be collected/acquired by using an acceleration sensor.

The first determining module 3920 is configured to determine whether the first limb is a dominant limb according to the first somatosensory information and reference information.

a) The first somatosensory information may be acceleration information of the first limb, e.g., first acceleration information. Correspondingly, the first determining module 3920 is configured to determine whether the first limb is a dominant limb according to the first acceleration information and reference information.

The first acceleration information may be acceleration values in a period of time, which may be acquired by using an acceleration sensor worn on the first limb according to a predetermined sampling frequency.

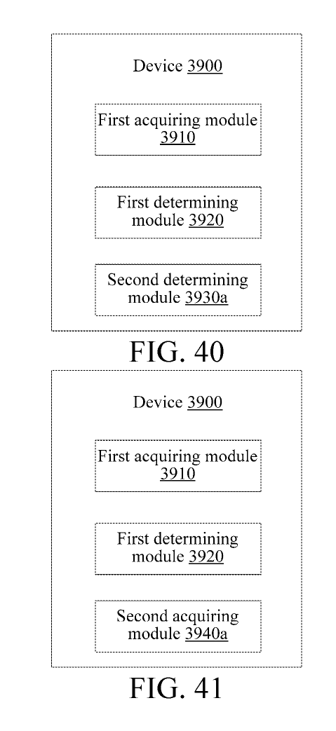

Referring to FIG. 40, in one implementation manner, the device 3900 further comprises a second determining module 3930a, configured to determine a threshold as the reference information according to an average absolute value of left-limb acceleration information and an average absolute value of right-limb acceleration information of the user.

The first determining module 3920 is configured to identify that the first limb is a dominant limb, in response to that an average absolute value of the first acceleration information is greater than the threshold; and determining that the first limb is not a dominant limb, in response to that the average absolute value of the first acceleration information is less than the threshold.

For example, the reference information may be a threshold determined according to an average absolute value of left-hand acceleration information and an average absolute value of right-hand acceleration information of the user. Assuming that the average absolute value of the left-hand acceleration information of the user falls within a first range Lₘᵢₙ, Lₘₐₓ), assuming that the average absolute value of the right-hand acceleration information of the user falls within a second range (Rₘᵢₙ, Rₘₐₓ), and assuming that the left hand of the user is a dominant hand, it is obtained that and it can be determined that the threshold is M, and Lₘᵢₙ>M>Rₘₐₓ. That is, the threshold M is a value between the first range and the second range.

Therefore, if the average absolute value of the first acceleration information is greater than the threshold M, it is considered that it falls within the first range, and the first limb is a dominant hand; if the average absolute value of the first acceleration information is less than the threshold M, it is considered that it falls within the second range, and the first limb is not a dominant hand.

It should be noted that, in this implementation manner, the reference information needs to be determined according to the left-limb acceleration information and the right-limb acceleration information of the user; therefore, the left-limb acceleration information and the right-limb acceleration information of the user need to be acquired in advance, for example, before formally using the dominant limb identification device, the user wears the device on the left hand for a period of time and then wears the device on the right hand for a period of time, so as to complete a training process.

In addition, to ensure the accuracy of identification, a sampling time for the first acceleration information should be long enough, for example, greater than a time threshold (for example, 10 minutes).

Referring to FIG. 41, in another implementation manner, the device 3900 further comprises a second acquiring module 3940a, configured to acquire second acceleration information of a second limb of the user as the reference information.

The first determining module 3920 is configured to identify that the first limb is a dominant limb, in response to that an average absolute value of the first acceleration information is greater than an average absolute value of the second acceleration information; and determine that the first limb is not a dominant limb, in response to that the average absolute value of the first acceleration information is less than the average absolute value of the second acceleration information.

In this implementation manner, the user does not need to perform training in advance, and for example, may separately acquire the first acceleration information of the first limb and the second acceleration information of the second limb of the user, and then compare the average absolute value of the first acceleration information with the average absolute value of the second acceleration information, so as to identify a dominant limb.

This implementation manner is advantageous in that the user does not need to complete a training process intentionally, and collection of the reference information is completed during normal use by the user; however, the device in this implementation manner may require acquiring acceleration information of, for example, both two hands, and is mainly applicable to scenarios in which the user needs to move the two hands at the same time, for example, the user wears smart gloves on the two hands to play an electronic game.

b) The first somatosensory information may be blood flow information of the first limb, e.g., first blood flow information. Correspondingly, the first determining module 3920 is configured to determine whether the first limb is a dominant limb according to the first blood flow information and reference information.

The blood flow information may be blood flow signals in a period of time, and may be acquired by using at least one blood flow sensor that is close to or in contact with the skin of the user. The first blood flow information is blood flow information acquired on the first limb.

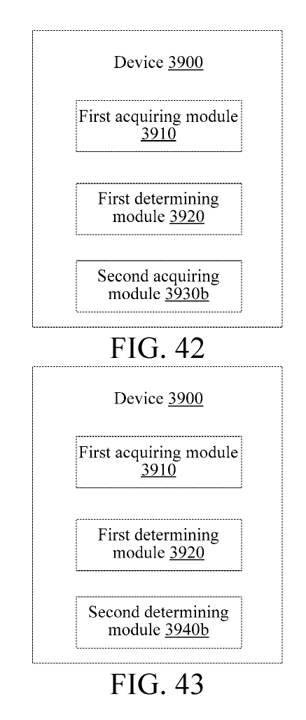

In one implementation manner, the reference information is second blood flow information of a second limb of the user, and referring to FIG. 42, the device 3900 further comprises a second acquiring module 3930b, configured to acquire the second blood flow information of the second limb of the user as the reference information.

Correspondingly, the first determining module 3920 is configured to determine that the first limb is a dominant limb, in response to that an average value of the first blood flow information is greater than an average value of the second blood flow information; and determine that the first limb is not a dominant limb, in response to that the average value of the first blood flow information is less than the average value of the second blood flow information.

In another implementation manner, the reference information of a threshold determined according to an average value of left-limb blood flow information and an average value of right-limb blood flow information of the user. Specifically, and referring to FIG. 43, the device 3900 further comprises a second determining module 3940b, configured to determine a threshold as the reference information according to an average value of left-limb blood flow information and an average value of right-limb blood flow information of the user.

Correspondingly, the first determining module 3920 is configured to determine that the first limb is a dominant limb, in response to that an average value of the first blood flow information is greater than the threshold; and determine that the first limb is not a dominant limb, in response to that the average value of the first blood flow information is less than the threshold.

For example, the second determining module 3940b may collect in advance, analyze and process the right-limb blood flow information and the left-limb blood flow information of the user. Assuming that the average value of the right-limb blood flow information falls within a first range (R_{bmin}, R_{bmax}), assuming that the average value of the left-limb blood flow information falls within a second range (L_{bmin}, L_{bmax}), and assuming that the right limb is a dominant limb, it is obtained that L_{bmax}<R_{bmin}, and it can be determined that the threshold is M_{b}, and L_{bmax}<M_{b}<R_{bmin}. That is, the threshold M_{b} is a value between the first range and the second range.

Therefore, if the average value of the first blood flow information is greater than the threshold M_{b}, it is considered that it falls within the first range, and the first limb is a dominant limb of the user; if the average value of the first blood flow information is less than the threshold M_{b}, it is considered that it falls within the second range, and the first limb is not a dominant limb of the user.

c) The first somatosensory information may be PPG information of the first limb, e.g., first PPG information. Correspondingly, the first determining module 3920 is configured to determine whether the first limb is a dominant limb according to the first PPG information and reference information.

The PPG information may be PPG signals in a period of time, and may be acquired by using at least one PPG sensor that is close to or in contact with the skin of the user. The PPG signal is a voltage change signal that reflects a change in blood volume caused by throbbing of artery vessels and is obtained by a photodetector when light is transmitted or reflected to pass through blood vessels in the human body. The first PPG information is PPG information acquired on the first limb.

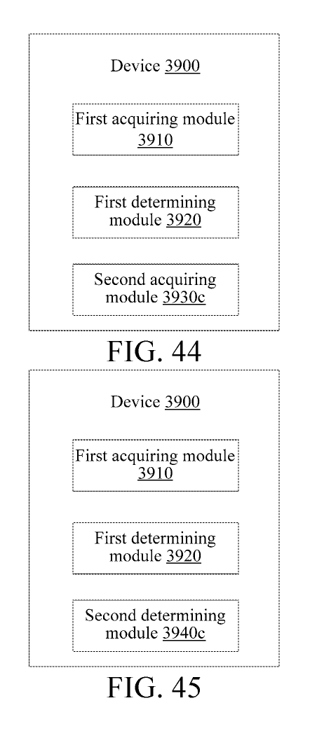

In one implementation manner, the reference information is second PPG information of a second limb of the user, and referring to FIG. 44, the device 3900 further comprises a second acquiring module 3930c, configured to acquire second PPG information of a second limb of the user as the reference information.

Correspondingly, the first determining module 3920 is configured to determine that the first limb is not a dominant limb, in response to that an average amplitude value of the first PPG information is greater than an average amplitude value of the second PPG information; and determine that the first limb is a dominant limb, in response to that the average amplitude value of the first PPG information is less than the average amplitude value of the second PPG information.

In another implementation manner, the reference information is a threshold determined according to an average amplitude value of left-limb PPG information and an average amplitude value of right-limb PPG information of the user. Specifically, and referring to FIG. 45, the device 3900 further comprises a second determining module 3940c, configured to determine a threshold as the reference information according to an average amplitude value of left-limb PPG information and an average amplitude value of right-limb PPG information of the user.

Correspondingly, the first determining module 3920 is configured to determine that the first limb is not a dominant limb, in response to that an average amplitude value of the first PPG information is greater than the threshold; and determine that the first limb is a dominant limb, in response to that the average amplitude value of the first PPG information is less than the threshold.

For example, the second determining module 3940c may collect in advance, analyze and process the right-limb PPG information and the left-limb PPG information of the user. Assuming that the average amplitude value of the right-limb PPG information falls within a first range (Rₚₘᵢₙ, Rₚₘₐₓ), assuming that the average amplitude value of the left-limb PPG information falls within a second range (Lₚₘᵢₙ, Lₚₘₐₓ), and assuming that the left limb is a dominant limb, it is obtained that Lₚₘₐₓ<Rₚₘᵢₙ, and it can be determined that the threshold is Mₚ, and Lₚₘₐₓ<Mₚ<Rₚₘᵢₙ. That is, the threshold Mₚ is a value between the first range and the second range.

Therefore, if the average amplitude value of the first PPG information is greater than the threshold Mₚ, it is considered that it falls within the first range, and the first limb is not a dominant limb of the user; if the average amplitude value of the first PPG information is less than the threshold Mₚ, it is considered that it falls within the second range, and the first limb is a dominant limb of the user.

d) The first somatosensory information may be temperature information of the first limb, e.g., first temperature information. Correspondingly, the first determining module 3920 is configured to determine whether the first limb is a dominant limb according to the first temperature information and reference information.

The first temperature information may be temperature signals in a period of time, and may be acquired by using at least one temperature sensor that is close to or in contact with the skin of the user. The first temperature information is temperature information acquired on the first limb.

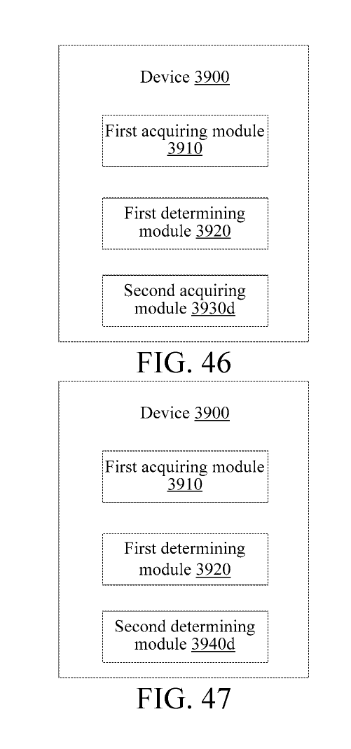

In one implementation manner, the reference information is second temperature information of a second limb of the user, and referring to FIG. 46, the device 3900 further comprises a second acquiring module 3930d, configured to acquire the second temperature information of the second limb of the user as the reference information.

Correspondingly, the first determining module 3920 is configured to determine that the first limb is a dominant limb, in response to that an average value of the first temperature information is greater than an average value of the second temperature information; and determine that the first limb is not a dominant limb, in response to that the average value of the first temperature information is less than the average value of the second temperature information.

In another implementation manner, the reference information is a threshold determined according to an average value of left-limb temperature information and an average value of right-limb temperature information of the user. Specifically, and referring to FIG. 47, the device 3900 further comprises a second determining module 3940d, configured to determine a threshold as the reference information according to an average value of left-limb temperature information and an average value of right-limb temperature information of the user.

Correspondingly, the first determining module 3920 is configured to determine that the first limb is a dominant limb, in response to that an average value of the first temperature information is greater than the threshold; and determine that the first limb is not a dominant limb, in response to that the average value of the first temperature information is less than the threshold.

For example, the second determining module 3940d may collect in advance, analyze and process the right-limb temperature information and the left-limb temperature information of the user. Assuming that the average value of the right-limb temperature information falls within a first range (Rₜₘᵢₙ, Rₜₘₐₓ), assuming that the average value of the left-limb temperature information falls within a second range (Lₜₘᵢₙ, Lₜₘₐₓ), and assuming that the right limb is a dominant limb, it is obtained that Lₜₘₐₓ<Rₜₘᵢₙ, and it can be determined that the threshold is Mₜ, and Lₜₘₐₓ<Mₜ<Rₜₘᵢₙ. That is, the threshold Mₜ is a value between the first range and the second range.

Therefore, if the average value of the first temperature information is greater than the threshold Mₜ, it is considered that it falls within the first range, and the first limb is a dominant limb of the user; if the average value of the first temperature information is less than the threshold Mₜ, it is considered that it falls within the second range, and the first limb is not a dominant limb of the user.

e) The first somatosensory information may be humidity information of the first limb, e.g., first humidity information. Correspondingly, the first determining module 3920 is configured to determine whether the first limb is a dominant limb according to the first humidity information and reference information.

The humidity information may be humidity signals in a period of time, and may be acquired by using at least one humidity sensor that is close to or in contact with the skin of the user. The first humidity information is humidity information acquired on the first limb.

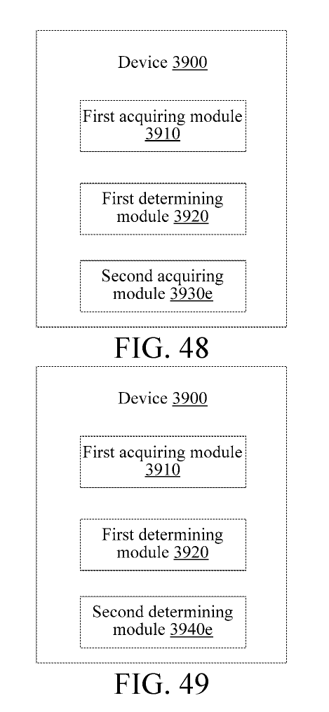

In one implementation manner, the reference information is second humidity information of a second limb of the user, and referring to FIG. 48, the device 3900 further comprises a second acquiring module 3930e, configured to acquire the second humidity information of the second limb of the user as the reference information.

Correspondingly, the first determining module 3920 is configured to determine that the first limb is a dominant limb, in response to that an average value of the first humidity information is greater than an average value of the second humidity information; and determine that the first limb is not a dominant limb, in response to that the average value of the first humidity information is less than the average value of the second humidity information.

In another implementation manner, the reference information is a threshold determined according to an average value of left-limb humidity information and an average value of right-limb humidity information of the user. Specifically, and referring to FIG. 49, the device 3900 further comprises a second determining module 3940e, configured to determine a threshold as the reference information according to an average value of left-limb humidity information and an average value of right-limb humidity information of the user.

Correspondingly, the first determining module 3920 is configured to determine that the first limb is a dominant limb, in response to that an average value of the first humidity information is greater than the threshold; and determine that the first limb is not a dominant limb, in response to that the average value of the first humidity information is less than the threshold.

For example, the second determining module 3940e may collect in advance, analyze and process the right-limb humidity information and the left-limb humidity information of the user. Assuming that the average value of the right-limb humidity information falls within a first range (Rₕₘᵢₙ, Rₕₘₐₓ), assuming that the average value of the left-limb humidity information falls within a second range (Lₕₘᵢₙ, Lₕₘₐₓ), and assuming that the right limb is a dominant limb, it is obtained that Lₕₘₐₓ<Rₕₘᵢₙ, and it can be determined that the threshold is Mₕ, and Lₕₘₐₓ<Mₕ<Rₕₘᵢₙ. That is, the threshold Mₕ is a value between the first range and the second range.

Therefore, if the average value of the first humidity information is greater than the threshold Mₕ, it is considered that it falls within the first range, and the first limb is a dominant limb of the user; if the average value of the first humidity information is less than the threshold Mₕ, it is considered that it falls within the second range, and the first limb is not a dominant limb of the user.

f) The first somatosensory information may be fat information of the first limb, e.g., first fat information. Correspondingly, the first determining module 3920 is configured to determine whether the first limb is a dominant limb according to the first fat information and reference information.

The fat information may be fat ratio information of a corresponding part of the body, and may be acquired by using at least one fat sensor that is close to or in contact with the skin of the user. The first fat information is fat ratio information acquired on the first limb.

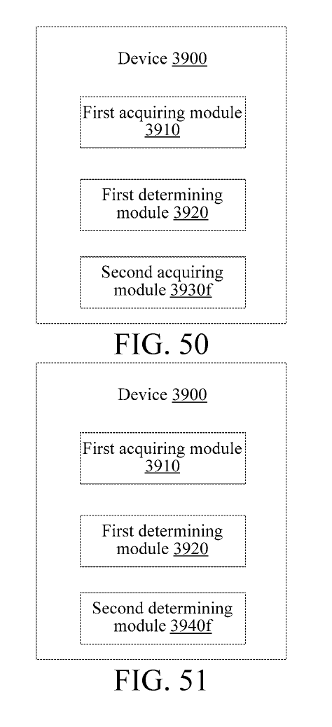

In one implementation manner, the reference information is second fat information of a second limb of the user, and referring to FIG. 50, the device 3900 further comprises a second acquiring module 3930f, configured to acquire the second fat information of the second limb of the user as the reference information.

Correspondingly, the first determining module 3920 is configured to determine that the first limb is a dominant limb, in response to that an average value of the first fat information is greater than an average value of the second fat information; and determine that the first limb is not a dominant limb, in response to that the average value of the first fat information is less than the average value of the second fat information.

In another implementation manner, the reference information is a threshold determined according to an average value of left-limb fat information and an average value of right-limb fat information of the user. Specifically, and referring to FIG. 51, the device 3900 further comprises a second determining module 3940f, configured to determine a threshold as the reference information according to an average value of left-limb fat information and an average value of right-limb fat information of the user.

Correspondingly, the first determining module 3920 is configured to determine that the first limb is a dominant limb, in response to that an average value of the first fat information is greater than the threshold; and determine that the first limb is not a dominant limb, in response to that the average value of the first fat information is less than the threshold.

For example, the second determining module 3940f may collect in advance, analyze and process the right-limb fat information and the left-limb fat information of the user. Assuming that the average value of the right-limb fat information falls within a first range (R_{fmin}, R_{fmax}), assuming that the average value of the left-limb fat information falls within a second range (L_{fmin}, L_{fmax}), and assuming that the right limb is a dominant limb, it is obtained that L_{fmax}<R_{fmin}, and it can be determined that the threshold is M_{f}, and L_{fmax}<M_{f}<R_{fmin}. That is, the threshold M_{f} is a value between the first range and the second range.

Therefore, if the average value of the first fat information is greater than the threshold M_{f}, it is considered that it falls within the first range, and the first limb is a dominant limb of the user; if the average value of the first fat information is less than the threshold M_{f}, it is considered that it falls within the second range, and the first limb is not a dominant limb of the user.

g) The first somatosensory information may be alkalinity/acidity information of the first limb, e.g., first alkalinity/acidity information. Correspondingly, the first determining module 3920 is configured to determine whether the first limb is a dominant limb according to the first alkalinity/acidity information and reference information.

The alkalinity/acidity information may be PH value information of a corresponding part of the body in a period of time, and may be acquired by using at least one alkalinity/acidity sensor that is close to or in contact with the skin of the user. The first alkalinity/acidity information is alkalinity/acidity information acquired on the first limb.

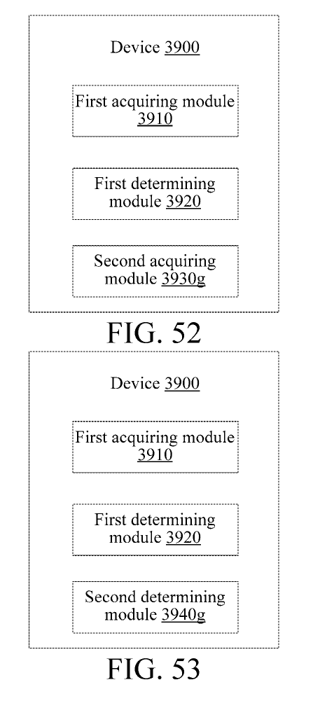

In one implementation manner, the reference information is second alkalinity/acidity information of a second limb of the user, and referring to FIG. 52, the device 3900 further comprises a second acquiring module 3930g, configured to acquire the second alkalinity/acidity information of the second limb of the user as the reference information.

Correspondingly, the first determining module 3920 is configured to determine that the first limb is a dominant limb, in response to that an average value of the first alkalinity/acidity information is greater than an average value of the second alkalinity/acidity information; and determine that the first limb is not a dominant limb, in response to that the average value of the first alkalinity/acidity information is less than the average value of the second alkalinity/acidity information.

In another implementation manner, the reference information is a threshold determined according to an average value of left-limb alkalinity/acidity information and an average value of right-limb alkalinity/acidity information of the user. Specifically, and referring to FIG. 53, the device 3900 further comprises a second determining module 3940g, configured to determine a threshold as the reference information according to an average value of left-limb alkalinity/acidity information and an average value of right-limb alkalinity/acidity information of the user.

Correspondingly, the first determining module 3920 is configured to determine that the first limb is a dominant limb, in response to that an average value of the first alkalinity/acidity information is greater than the threshold; and determine that the first limb is not a dominant limb, in response to that the average value of the first alkalinity/acidity information is less than the threshold.

For example, the second determining module 3940g may collect in advance, analyze and process the right-limb alkalinity/acidity information and the left-limb alkalinity/acidity information of the user. Assuming that the average value of the right-limb alkalinity/acidity information falls within a first range (Rₚₘᵢₙ, Rₚₘₐₓ), assuming that the average value of the left-limb alkalinity/acidity information falls within a second range (Lₚₘᵢₙ, Lₚₘₐₓ), and assuming that the right limb is a dominant limb, it is obtained that Lₚₘₐₓ<Rₚₘᵢₙ, and it can be determined that the threshold is Mₚ, and Lₚₘₐₓ<Mₚ<Rₚₘᵢₙ. That is, the threshold Mₚ is a value between the first range and the second range.

Therefore, if the average value of the first alkalinity/acidity information is greater than the threshold Mₚ, it is considered that it falls within the first range, and the first limb is a dominant limb of the user; if the average value of the first alkalinity/acidity information is less than the threshold Mₚ, it is considered that it falls within the second range, and the first limb is not a dominant limb of the user.

h) The first somatosensory information may be skin conductance information of the first limb, e.g., first skin conductance information. Correspondingly, the first determining module 3920, configured to determine (or identify) whether the limb is a dominant limb according to the skin conductance information and reference information.

The skin conductance information may be eigenvalues of the skin conductance level (skin conductance level), and may be acquired by using a skin conductance sensor that is in contact with the skin of the user. Generally all existing smart bracelets, smart wristbands and the like are equipped with the skin conductance sensor; therefore, the device of the present application does not increase the hardware costs of the existing wearable device.

The reference information may be a threshold determined according to left-limb skin conductance information and right-limb skin conductance information of the user, or may be the left-limb skin conductance information or the right-limb skin conductance information of the user. Different cases are described in detail below respectively.

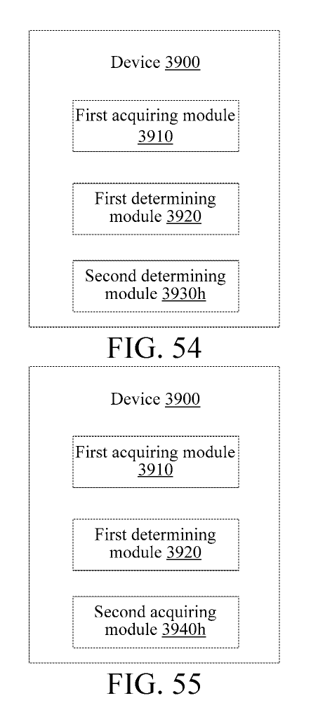

Referring to FIG. 54, in one implementation manner, the device 3900 further comprises a second determining module 3930h, configured to determine a threshold as the reference information according to an average amplitude value of left-limb skin conductance information and an average amplitude value of right-limb skin conductance information of the user.

In this implementation manner, the first determining module 3920 is configured to determine (or identify) that the limb is a dominant limb, in response to that an average amplitude value of the first skin conductance information is less than the threshold; and determine (or identify) that the limb is not a dominant limb, in response to that the average amplitude value of the skin conductance information is not less than the threshold.

For example, the reference information may be a threshold determined according to left-hand skin conductance information and right-hand skin conductance information of the user. Assuming that an average amplitude value of the left-hand skin conductance information of the user falls within a first range (Lₘᵢₙ, assuming that an average amplitude value of the right-hand skin conductance information of the user falls within a second range (Rₘᵢₙ, Rₘₐₓ), and assuming that the left hand of the user is a dominant hand, it is obtained that Lₘₐₓ<Rₘᵢₙ, and the first determining module 3920 can determine that the threshold is M, and Lₘₐₓ<M<Rₘᵢₙ. That is, the threshold M is a value between the first range and the second range.

Therefore, for the first determining module 3920, if the average amplitude value of the skin conductance information is less than the threshold M, it is considered that it falls within the first range, and the limb is a dominant hand; if the average amplitude value of the skin conductance information is not less than the threshold M, it is considered that it falls within the second range, and the limb is not a dominant hand.

It should be noted that, in this implementation manner, the reference information needs to be determined according to the left-limb skin conductance information and the right-limb skin conductance information of the user; therefore, the left-limb skin conductance information and the right-limb skin conductance information of the user need to be acquired in advance, for example, before formally using the dominant limb determination device, the user wears the device on the left hand for a period of time and then wears the device on the right hand for a period of time, so as to complete a training process.

In another implementation manner, the reference information is left-limb skin conductance information or right-limb skin conductance information of the user. The first determining module 3920 is configured to determine (or identify) that the limb is a dominant limb, in response to that the skin conductance information is statistically significantly different from the reference information and an average amplitude value of the skin conductance information is less than an average amplitude value of the reference information; and determine (or identify) that the limb is not a dominant limb, in response to that the skin conductance information is statistically significantly different from the reference information and the average amplitude value of the skin conductance information is greater than the average amplitude value of the reference information.

In this implementation manner, the user does not need to perform training in advance, and for example, in response to that the left hand or the right hand of the user wears the dominant limb determination device for the first time, the first skin conductance information is acquired, which is used as the reference information; in response to that the user wears the dominant limb determination device again after a period of time (for example, on the next day), the second skin conductance information is acquired. The first skin conductance information and the second skin conductance information may be skin conductance information of a same hand or may respectively be skin conductance information of two hands, and according to the principle for determining (or identifying) a dominant hand in the foregoing method embodiment, the dominant hand of the user can be determined (or identified) only when the first skin conductance information and the second skin conductance information are respectively skin conductance information of two hands.

Therefore, in the first determining module 3920, the condition that the skin conductance information is statistically significantly different from the reference information needs to be satisfied first, indicating that the skin conductance information and the reference information are respectively skin conductance information of two hands, and then the average amplitude value of the skin conductance information may be compared with the average amplitude value of the reference information, so as to complete the determination (or identification) process.

This implementation manner is advantageous in that the user does not need to complete a training process intentionally, and collection of the reference information is completed during normal use by the user; however, the device in this implementation manner still requires acquiring skin conductance information of both two hands in turn, and is mainly applicable to scenarios in which the user wears a wearable device on two hands alternately.

In addition, it should be understood by a person skilled in the art that when a user has worn the dominant limb determination (or identification) device on two hands, the device 3900 can properly select skin conductance information of one hand as the reference information, to ensure that the skin conductance information is statistically significantly different from the reference information, so as to complete the determination (or identification) process.

Referring to FIG. 55, in another implementation manner, the device 3900 further comprises a second acquiring module 3940h, configured to acquire second skin conductance information of a second limb of the user as the reference information.

Correspondingly, the first determining module 3920 is configured to determine that the first limb is not a dominant limb, in response to that an average amplitude value of the first skin conductance information is greater than an average amplitude value of the second skin conductance information; and determine that the first limb is a dominant limb, in response to that the average amplitude value of the first skin conductance information is less than the average amplitude value of the second skin conductance information.

i) The first somatosensory information may be electromyogram information of the first limb, e.g., first electromyogram information. Correspondingly, the first determining module 3920, configured to determine (or identify) whether the first limb is a dominant limb according to the first electromyogram information and reference information.

The electromyogram information may be electromyogram signals in a period of time, and may be acquired by using a set of electromyogram sensors that is in contact with the skin of the user. The set of electromyogram sensors comprises one or more electromyogram sensors. The electromyogram sensor may comprise an electrode, an amplifier circuit, and so on.

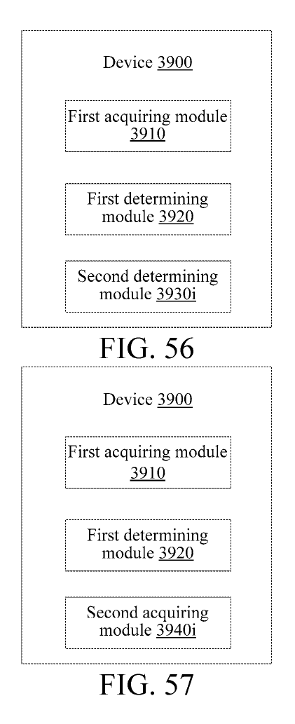

Referring to FIG. 56, in one implementation manner, the device 3900 further comprises a second determining module 3930i, configured to determine a threshold as the reference information according to an average amplitude value of left-limb electromyogram information and an average amplitude value of right-limb electromyogram information of the user.

The first determining module 3920 is configured to determine that the first limb is a dominant limb, in response to that an average amplitude value of the first electromyogram information is greater than the threshold and determine that the first limb is not a dominant limb, in response to that the average amplitude value of the first electromyogram information is less than the threshold.

For example, the second determining module 3930i may determine the threshold as the reference information according to an average amplitude value of left-hand electromyogram information and an average amplitude value of the right-hand electromyogram information of the user. Assuming that the average amplitude value of the left-hand electromyogram information of the user falls within a first range (Lₘᵢₙ, Lₘₐₓ), assuming that the average amplitude value of the right-hand electromyogram information of the user falls within a second range (Rₘᵢₙ, Rₘₐₓ), and assuming that the left hand of the user is a dominant hand, it is obtained that and it can be determined that the threshold is M, and Lₘᵢₙ>M>Rₘₐₓ. That is, the threshold M is a value between the first range and the second range.

Therefore, for the first determining module 3920, if the average value of the electromyogram information is greater than the threshold M, it is considered that it falls within the first range, and it is determined (or identified) that the limb is a dominant hand; if the average value of the electromyogram information is less than the threshold M, it is considered that it falls within the second range, and it is determined (or identified) that the limb is not a dominant hand.

It should be noted that, in this implementation manner, the reference information needs to be determined according to the left-limb electromyogram information and the right-limb electromyogram information of the user; therefore, the first determining module 30 needs to acquire the left-limb electromyogram information and the right-limb electromyogram information of the user in advance, for example, before formally using the dominant limb determination device, the user wears the device on the left hand for a period of time and then wears the device on the right hand for a period of time, so as to complete a training process.

Referring to FIG. 57, in another implementation manner, the device 3900 further comprises a second acquiring module 3940i, configured to acquire second electromyogram information of a second limb of the user as the reference information.

The first determining module 3920 is configured to determine that the first limb is a dominant limb, in response to that an average amplitude value of the first electromyogram information is greater than an average amplitude value of the second electromyogram information; and determine that the first limb is not a dominant limb, in response to that the average amplitude value of the first electromyogram information is less than the average amplitude value of the second electromyogram information.

In this implementation manner, the user does not need to perform training in advance, and for example, may separately acquire the first electromyogram information of the first limb and the second electromyogram information of the second limb of the user, and then compare the average amplitude value of the first electromyogram information with the average amplitude value of the second electromyogram information, so as to determine (or identify) a dominant limb.

This implementation manner is advantageous in that the user does not need to complete a training process intentionally, and collection of the reference information is completed during normal use by the user; however, the method in this implementation manner may require acquiring electromyogram information of, for example, both two hands, and is mainly applicable to scenarios in which the user needs to use the two hands at the same time, for example, the user wears smart gloves on the two hands to play an electronic game.

j) The first somatosensory information may be bio-impedance information of the first limb, e.g., first bio-impedance information. Correspondingly, the first determining module 3920 is configured to determine whether the first limb is a dominant limb according to the first bio-impedance information and reference information.

The bio-impedance information may be bio-impedance signals in a period of time, and may be acquired by using at least one bio-impedance sensor that is close to or in contact with the skin of the user. The first bio-impedance information is bio-impedance information acquired on the first limb.

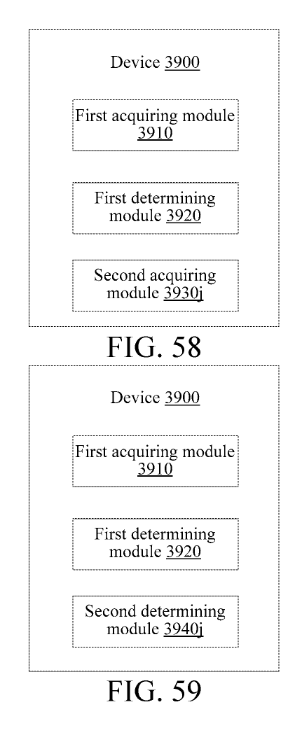

In one implementation manner, the reference information is second bio-impedance information of a second limb of the user, and referring to FIG. 58, the device 3900 further comprises a second acquiring module 3930j, configured to acquire the second bio-impedance information of the second limb of the user as the reference information.

Correspondingly, the first determining module 3920 is configured to determine that the first limb is a dominant limb, in response to that an average amplitude value of the first bio-impedance information is greater than an average amplitude value of the second bio-impedance information; and determine that the first limb is not a dominant limb, in response to that the average amplitude value of the first bio-impedance information is less than the average amplitude value of the second bio-impedance information.

In another implementation manner, the reference information is a threshold determined according to an average amplitude value of left-limb bio-impedance information and an average amplitude value of right-limb bio-impedance information of the user. Specifically, and referring to FIG. 59, the device 3900 further comprises a second determining module 3940j, configured to determine a threshold as the reference information according to an average amplitude value of left-limb bio-impedance information and an average amplitude value of right-limb bio-impedance information of the user.

Correspondingly, the first determining module 3920 is configured to determine that the first limb is a dominant limb, in response to that an average amplitude value of the first bio-impedance information is greater than the threshold; and determine that the first limb is not a dominant limb, in response to that the average amplitude value of the first bio-impedance information is less than the threshold.

For example, the second determining module 3940j may collect in advance, analyze and process the right-limb bio-impedance information and the left-limb bio-impedance information of the user. Assuming that the average amplitude value of the right-limb bio-impedance information falls within a first range (Rᵣₘᵢₙ, Rᵣₘₐₓ), assuming that the average amplitude value of the left-limb bio-impedance information falls within a second range (Lᵣₘᵢₙ, Lᵣₘₐₓ), and assuming that the right limb is a dominant limb, it is obtained that Lᵣₘₐₓ<Rᵣₘᵢₙ, and it can be determined that the threshold is Mᵣ, and Lᵣₘₐₓ<Mᵣ<Rᵣₘᵢₙ. That is, the threshold Mᵣ is a value between the first range and the second range.

Therefore, if the average amplitude value of the first bio-impedance information is greater than the threshold Mᵣ, it is considered that it falls within the first range, and the first limb is a dominant limb of the user; if the average amplitude value of the first bio-impedance information is less than the threshold Mᵣ, it is considered that it falls within the second range, and the first limb is not a dominant limb of the user.

k) The first somatosensory information may be blood oxygen information of the first limb, e.g., first blood oxygen information. Correspondingly, the first determining module 3920 is configured to determine whether the first limb is a dominant limb according to the first blood oxygen information and reference information.

The blood oxygen information may be blood oxygen saturation information in a period of time, and may be acquired by using at least one blood oxygen sensor that is close to or in contact with the skin of the user. The first blood oxygen information is blood oxygen information acquired on the first limb.

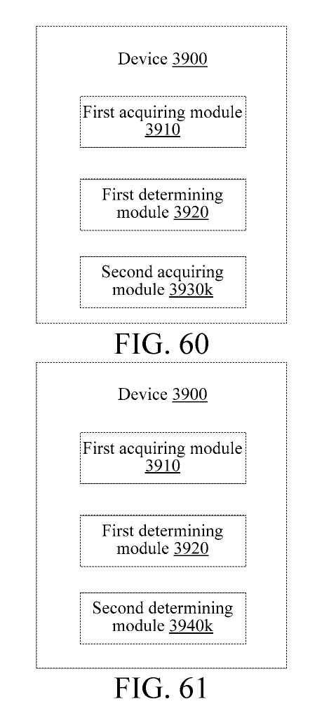

In one implementation manner, the reference information is second blood oxygen information of a second limb of the user, and referring to FIG. 60, the device 3900 further comprises a second acquiring module 3930k, configured to acquire the second blood oxygen information of the second limb of the user as the reference information.

Correspondingly, the first determining module 3920 is configured to determine that the first limb is not a dominant limb, in response to that an average value of the first blood oxygen information is greater than an average value of the second blood oxygen information; and determine that the first limb is a dominant limb, in response to that the average value of the first blood oxygen information is less than the average value of the second blood oxygen information.

In another implementation manner, the reference information is a threshold determined according to an average value of left-limb blood oxygen information and an average value of right-limb blood oxygen information of the user. Specifically, and referring to FIG. 61, the device 3900 further comprises a second determining module 3940k, configured to determine a threshold as the reference information according to an average value of left-limb blood oxygen information and an average value of right-limb blood oxygen information of the user.

Correspondingly, the first determining module 3920 is configured to determine that the first limb is not a dominant limb, in response to that an average value of the first blood oxygen information is greater than the threshold; and determine that the first limb is a dominant limb, in response to that the average value of the first blood oxygen information is less than the threshold.

For example, the second determining module 3940k may collect in advance, analyze and process the right-limb blood oxygen information and the left-limb blood oxygen information of the user. Assuming that the average value of the right-limb blood oxygen information falls within a first range (Rₒₘᵢₙ, Rₒₘₐₓ), assuming that the average value of the left-limb blood oxygen information falls within a second range (Lₒₘᵢₙ, Lₒₘₐₓ), and assuming that the left limb is a dominant limb, it is obtained that Lₒₘₐₓ<Rₒₘᵢₙ, and it can be determined that the threshold is Mₒ, and Lₒₘₐₓ<Mₒ<Rₒₘᵢₙ. That is, the threshold Mₒ is a value between the first range and the second range.

Therefore, if the average value of the first blood oxygen information is greater than the threshold Mₒ, it is considered that it falls within the first range, and the first limb is not a dominant limb of the user; if the average value of the first blood oxygen information is less than the threshold Mₒ, it is considered that it falls within the second range, and the first limb is a dominant limb of the user.

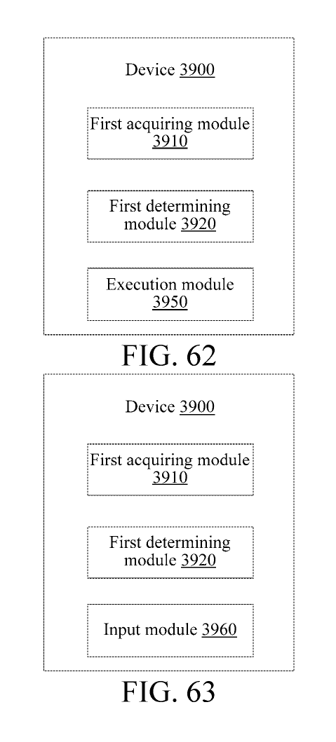

Referring to FIG. 62, in one implementation manner, the device 3900 further comprises an execution module 3950, configured to perform an operation according to an identification result.

For example, it is learned according to the identification result that the smart wristband is worn on the dominant hand of the user, and since the dominant hand is often needed during work, a prompt may be provided when the arm of the dominant hand of the user moves frequently, to remind the user to take care of the wristband to avoid collision.

For another example, the user wears smart gloves on two hands to play an electronic shooting game, and if the gloves identify that the left hand of the user is the dominant hand, the execution module 3950 can automatically control the gaming machine to switch to a right-hand gun holding mode, so as to better adapt to use habits of the user.

Referring to FIG. 63, in one implementation manner, the device 3900 further comprises an input module 3960, configured to receive input information of the user.

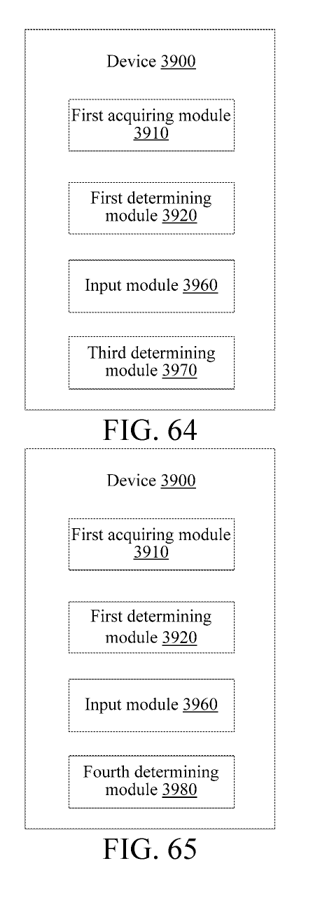

Referring to FIG. 64, in one implementation manner, the input information is dominant-limb information, and the device 3900 further comprises a third determining module 3970, configured to determine whether the first limb is a left limb or a right limb according to the input information and an identification result.

For example, if the dominant-limb information indicates that the user is left-handed, and the identification result indicates that the first limb is a dominant hand, it may be determined that the first limb is the left hand.

After it is determined that the first limb is the left limb or the right limb, settings such as a display interface of the wearable device worn on the first limb of the user can be adaptively adjusted, or settings such as a display interface of a device such as a smart phone held by the first limb may further be adjusted.

For example, in the method, assuming that it is determined that the first limb is the left hand of the user, an unlock gesture for a smart watch worn on the first limb may be set to be sliding from left to right, and a smart phone may be set to display consonants on the left half of the screen and display vowels on the right half of the screen.

Referring to FIG. 65, in another implementation manner, the input information comprises whether the first limb is a left hand or a right hand, and the device 3900 further comprises a fourth determining module 3980, configured to determine whether the user is left-handed or right-handed according to the input information and an identification result.

For example, the input information shows that the first limb is the left hand, and the identification result indicates that the first limb is a dominant hand, it may be determined that the user is left-handed. The input information may be a voice input, a text input, or the like, or may be a motion input. For example, when the user wears a left-hand glove on one hand, it is equivalent to inputting that the one hand is the left hand.

One application scenario of the dominant limb identification method and device according to the embodiments of the present application may be as follow: A left-handed user wears smart gloves on two hands to play an electronic game. At the beginning, the smart glove on the left hand collects electromyogram information of five fingers of the left hand at a predetermined frequency within a period of time of, for example, 10 minutes, and the smart glove on the right hand collects electromyogram information of five fingers of the right hand at the same frequency. Then, an average amplitude of the left-hand electromyogram information and an average amplitude of the right-hand electromyogram information are calculated respectively. The result shows that the average amplitude of the left-hand electromyogram information is generally greater than the average amplitude of the right-hand electromyogram information. Therefore, it is identified that the left hand is the dominant hand, and accordingly control buttons on the display screen are set on the side of the left hand, to make it convenient to use for the user, thereby improving user experience.

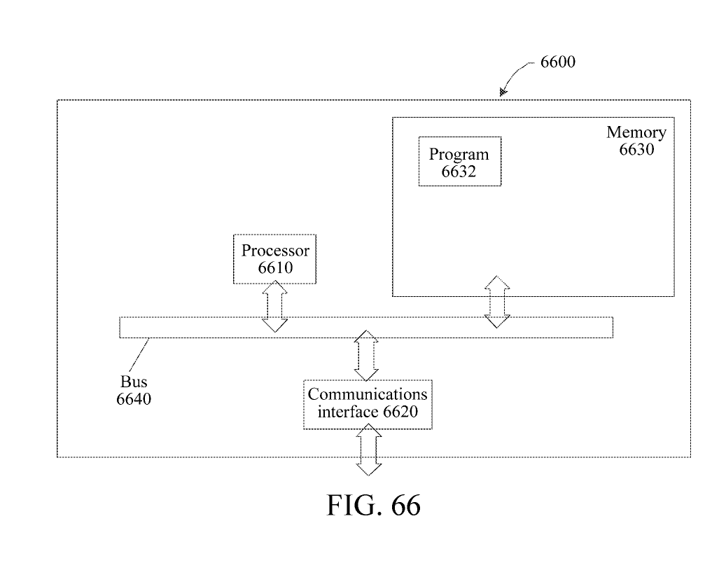

A hardware structure of an exemplary dominant hand identification device according to one embodiment of the present application is shown in FIG. 66. Specific embodiments of the present application are not intended to limit the specific implementation of the dominant hand identification device. Referring to FIG. 66, the device 6600 may comprise a processor 6610, a communications interface 6620, a memory 6630, and a communications bus 6640.

The processor 6610, the communications interface 6620, and the memory 6630 communicate with each other by using the communications bus 6640.

The communications interface 6620 is configured to communicate with another network element.

The processor 6610 is configured to execute a program 6632. Specifically, the processor 6610 can perform relevant steps in the foregoing method embodiment shown in FIG. 1.

Specifically, the program 6632 may comprise program code, where the program code comprises computer operation instructions.

The processor 6610 may be a central processing unit (CPU), an application specific integrated circuit (ASIC), or one or more integrated circuits configured to implement the embodiments of the present application.

The memory 6630 is configured to store the program 6632. The memory 6630 may comprise a high-speed random access memory (RAM memory), and may also comprise a non-volatile memory, such as at least one magnetic disk memory. The program 6632 may specifically perform the following steps:

Acquiring first somatosensory information of a first limb of a user; and

Determining whether the first limb is a dominant limb according to the first somatosensory information and reference information.

For the specific implementation of the steps in the program 6632, reference may be made to the corresponding steps or modules in the foregoing embodiments, which are not described herein again. It may be clearly understood by a person skilled in the art that, for the purpose of convenient and brief description, reference may be made to the description of corresponding procedures in the foregoing method embodiments for detailed working procedures of the foregoing devices and modules, and details are not described herein again.

A person of ordinary skill in the art may be aware that, units and method steps of the examples that are described in conjunction with the embodiments disclosed in this specification may be implemented by electronic hardware or a combination of computer software and electronic hardware. Whether the functions are performed by hardware or software depends on particular applications and design constraint conditions of the technical solution. A person skilled in the art may use different methods to implement the described functions for each particular application, but it should not be considered that the implementation goes beyond the scope of the present application.

When the functions are implemented in a form of a software functional unit and sold or used as an independent product, the functions may be stored in a computer-readable storage medium. Based on such an understanding, the technical solutions of the present application essentially, or the part contributing to the prior art, or a part of the technical solutions may be implemented in a form of a software product. The computer software product is stored in a storage medium and comprises several instructions for instructing a computer device (which may be a personal computer, a controller, a network device, or the like) to perform all or a part of the steps of the methods described in the embodiments of the present application. The foregoing storage medium comprises: any medium that can store program code, such as a USB flash drive, a removable hard disk, a read-only memory (ROM), a random access memory (RAM), a magnetic disk, or an optical disc.

The foregoing implementation manners are merely used to describe the present application, but are not intended to limit the present application. A person of ordinary skill in the art may further make various variations and modifications without departing from the spirit and scope of the present application. Therefore, all the equivalent technical solutions also fall within the scope of the present application, and the patent protection scope of the present application should be subject to the claims.

What is claimed is:

1. A dominant limb identification method, comprising:
acquiring first somatosensory information of a first limb of a user; and
determining whether the first limb is a dominant limb according to the first somatosensory information and reference information.

2. The method of claim 1, wherein the first somatosensory information is first acceleration information.

3. The method of claim 2, wherein the reference information is a threshold determined according to an average absolute value of left-limb acceleration information and an average absolute value of right-limb acceleration information of the user.

4. The method of claim 3, wherein determining whether the first limb is a dominant limb according to the first somatosensory information and reference information comprises:
determining that the first limb is a dominant limb when an average absolute value of the first acceleration information is greater than the threshold; and
determining that the first limb is not a dominant limb when the average absolute value of the first acceleration information is less than the threshold.

5. The method of claim 2, wherein the reference information is second acceleration information of a second limb of the user.

6. The method of claim 5, wherein determining whether the first limb is a dominant limb according to the first somatosensory information and reference information comprises:
determining that the first limb is a dominant limb when an average absolute value of the first acceleration information is greater than an average absolute value of the second acceleration information; and
determining that the first limb is not a dominant limb when the average absolute value of the first acceleration information is less than the average absolute value of the second acceleration information.

7. The method of claim 1, wherein the first somatosensory information is first blood flow information.

8. The method of claim 7, wherein the reference information is a threshold determined according to an average value of left-limb blood flow information and an average value of right-limb blood flow information of the user.

9. The method of claim 8, wherein determining whether the first limb is a dominant limb according to the first somatosensory information and reference information comprises:
determining that the first limb is a dominant limb when an average value of the first blood flow information is greater than the threshold; and
determining that the first limb is not a dominant limb when the average value of the first blood flow information is less than the threshold.

10. The method of claim 7, further comprising:
acquiring second blood flow information of a second limb of the user as the reference information.

11. The method of claim 10, wherein determining whether the first limb is a dominant limb according to the first somatosensory information and reference information comprises:
determining that the first limb is a dominant limb when an average value of the first blood flow information is greater than an average value of the second blood flow information; and
determining that the first limb is not a dominant limb when the average value of the first blood flow information is less than the average value of the second blood flow information.

12. The method of claim 1, wherein the first somatosensory information is first photoplethysmography (PPG) information.

13. The method of claim 12, wherein the reference information is a threshold determined according to an average amplitude value of left-limb PPG information and an average amplitude value of right-limb PPG information of the user.

14. The method of claim 13, wherein determining whether the first limb is a dominant limb according to the first somatosensory information and reference information comprises:
determining that the first limb is not a dominant limb when an average amplitude value of the first PPG information is greater than the threshold; and
determining that the first limb is a dominant limb when the average amplitude value of the first PPG information is less than the threshold.

15. The method of claim 12, further comprising:
acquiring second PPG information of a second limb of the user as the reference information.

16. The method of claim 15, wherein determining whether the first limb is a dominant limb according to the first somatosensory information and reference information comprises:
determining that the first limb is not a dominant limb when an average amplitude value of the first PPG information is greater than an average amplitude value of the second PPG information; and
determining that the first limb is a dominant limb when the average amplitude value of the first PPG information is less than the average amplitude value of the second PPG information.

17. The method of claim 1, wherein the first somatosensory information is first temperature information.

18. The method of claim 17, wherein the reference information is a threshold determined according to an average value of left-limb temperature information and an average value of right-limb temperature information of the user.

19. The method of claim 18, wherein determining whether the first limb is a dominant limb according to the first somatosensory information and reference information comprises:
determining that the first limb is a dominant limb when an average value of the first temperature information is greater than the threshold; and
determining that the first limb is not a dominant limb when the average value of the first temperature information is less than the threshold.

20. The method of claim 17, further comprising:
acquiring second temperature information of a second limb of the user as the reference information.

21. The method of claim 20, wherein determining whether the first limb is a dominant limb according to the first somatosensory information and reference information comprises:
determining that the first limb is a dominant limb when an average value of the first temperature information is greater than an average value of the second temperature information; and
determining that the first limb is not a dominant limb when the average value of the first temperature information is less than the average value of the second temperature information.

22. The method of claim 1, wherein the first somatosensory information is first humidity information.

23. The method of claim 22, wherein the reference information is a threshold determined according to an average value of left-limb humidity information and an average value of right-limb humidity information of the user.

24. The method of claim 23, wherein determining whether the first limb is a dominant limb according to the first somatosensory information and reference information comprises:
determining that the first limb is a dominant limb when an average value of the first humidity information is greater than the threshold; and
determining that the first limb is not a dominant limb when the average value of the first humidity information is less than the threshold.

25. The method of claim 22, further comprising:
acquiring second humidity information of a second limb of the user as the reference information.

26. The method of claim 25, wherein determining whether the first limb is a dominant limb according to the first somatosensory information and reference information comprises:
determining that the first limb is a dominant limb when an average value of the first humidity information is greater than an average value of the second humidity information; and
determining that the first limb is not a dominant limb when the average value of the first humidity information is less than the average value of the second humidity information.

27. The method of claim 1, wherein the first somatosensory information is first fat information.

28. The method of claim 27, wherein the reference information is a threshold determined according to an average value of left-limb fat information and an average value of right-limb fat information of the user.

29. The method of claim 28, wherein determining whether the first limb is a dominant limb according to the first somatosensory information and reference information comprises:
determining that the first limb is a dominant limb when an average value of the first fat information is greater than the threshold; and
determining that the first limb is not a dominant limb when the average value of the first fat information is less than the threshold.

30. The method of claim 27, further comprising:
acquiring second fat information of a second limb of the user as the reference information.

31. The method of claim 30, wherein determining whether the first limb is a dominant limb according to the first somatosensory information and reference information comprises:
determining that the first limb is a dominant limb when an average value of the first fat information is greater than an average value of the second fat information; and
determining that the first limb is not a dominant limb when the average value of the first fat information is less than the average value of the second fat information.

32. The method of claim 1, wherein the first somatosensory information is first alkalinity/acidity information.

33. The method of claim 32, wherein the reference information is a threshold determined according to an average value of left-limb alkalinity/acidity information and an average value of right-limb alkalinity/acidity information of the user.

34. The method of claim 33, wherein determining whether the first limb is a dominant limb according to the first somatosensory information and reference information comprises:
determining that the first limb is a dominant limb when an average value of the first alkalinity/acidity information is greater than the threshold; and
determining that the first limb is not a dominant limb when the average value of the first alkalinity/acidity information is less than the threshold.

35. The method of claim 32, further comprising:
acquiring second alkalinity/acidity information of a second limb of the user as the reference information.

36. The method of claim 35, wherein determining whether the first limb is a dominant limb according to the first somatosensory information and reference information comprises:
determining that the first limb is a dominant limb when an average value of the first alkalinity/acidity information is greater than an average value of the second alkalinity/acidity information; and
determining that the first limb is not a dominant limb when the average value of the first alkalinity/acidity information is less than the average value of the second alkalinity/acidity information.

37. The method of claim 1, wherein the first somatosensory information is first skin conductance information.

38. The method of claim 37, wherein the reference information is a threshold determined according to an average amplitude value of left-limb skin conductance information and an average amplitude value of right-limb skin conductance information of the user.

39. The method of claim 38, wherein determining whether the first limb is a dominant limb according to the first somatosensory information and reference information comprises:
determining that the first limb is not a dominant limb when an average amplitude value of the first skin conductance information is greater than the threshold; and
determining that the first limb is a dominant limb when the average amplitude value of the first skin conductance information is less than the threshold.

40. The method of claim 37, further comprising:
acquiring second skin conductance information of a second limb of the user as the reference information.

41. The method of claim 40, wherein determining whether the first limb is a dominant limb according to the first somatosensory information and reference information comprises:
determining that the first limb is not a dominant limb when an average amplitude value of the first skin conductance information is greater than an average amplitude value of the second skin conductance information; and
determining that the first limb is a dominant limb when the average amplitude value of the first skin conductance information is less than the average amplitude value of the second skin conductance information.

42. The method of claim 1, wherein the first somatosensory information is first electromyogram information.

43. The method of claim 42, wherein the reference information is a threshold determined according to an average amplitude value of left-limb electromyogram information and an average amplitude value of right-limb electromyogram information of the user.

44. The method of claim 43, wherein determining whether the first limb is a dominant limb according to the first somatosensory information and reference information comprises:
determining that the first limb is a dominant limb when an average amplitude value of the first electromyogram information is greater than the threshold; and
determining that the first limb is not a dominant limb when the average amplitude value of the first electromyogram information is less than the threshold.

45. The method of claim 42, further comprising:
acquiring second electromyogram information of a second limb of the user as the reference information.

46. The method of claim 45, wherein determining whether the first limb is a dominant limb according to the first somatosensory information and reference information comprises:
determining that the first limb is a dominant limb when an average amplitude value of the first electromyogram information is greater than an average amplitude value of the second electromyogram information; and
determining that the first limb is not a dominant limb when the average amplitude value of the first electromyogram information is less than the average amplitude value of the second electromyogram information.

47. The method of claim 1, wherein the first somatosensory information is first bio-impedance information.

48. The method of claim 47, wherein the reference information is a threshold determined according to an average amplitude value of left-limb bio-impedance information and an average amplitude value of right-limb bio-impedance information of the user.

49. The method of claim 48, wherein determining whether the first limb is a dominant limb according to the first somatosensory information and reference information comprises:
determining that the first limb is a dominant limb when an average amplitude value of the first bio-impedance information is greater than the threshold; and
determining that the first limb is not a dominant limb when the average amplitude value of the first bio-impedance information is less than the threshold.

50. The method of claim 47, further comprising:
acquiring second bio-impedance information of a second limb of the user as the reference information.

51. The method of claim 50, wherein determining whether the first limb is a dominant limb according to the first somatosensory information and reference information comprises:
determining that the first limb is a dominant limb when an average amplitude value of the first bio-impedance information is greater than an average amplitude value of the second bio-impedance information; and
determining that the first limb is not a dominant limb when the average amplitude value of the first bio-impedance information is less than the average amplitude value of the second bio-impedance information.

52. The method of claim 1, wherein the first somatosensory information is first blood oxygen information.

53. The method of claim 52, wherein the reference information is a threshold determined according to an average value of left-limb blood oxygen information and an average value of right-limb blood oxygen information of the user.

54. The method of claim 53, wherein determining whether the first limb is a dominant limb according to the first somatosensory information and reference information comprises:
determining that the first limb is not a dominant limb when an average value of the first blood oxygen information is greater than the threshold; and
determining that the first limb is a dominant limb when the average value of the first blood oxygen information is less than the threshold.

55. The method of claim 52, further comprising:
acquiring second blood oxygen information of a second limb of the user as the reference information.

56. The method of claim 55, wherein determining whether the first limb is a dominant limb according to the first somatosensory information and reference information comprises:
determining that the first limb is not a dominant limb when an average value of the first blood oxygen information is greater than an average value of the second blood oxygen information; and
determining that the first limb is a dominant limb when the average value of the first blood oxygen information is less than the average value of the second blood oxygen information.

57. The method of claim 1, further comprising:
performing an operation according to a determination result.

58. The method of claim 1, further comprising:
receiving input information from the user.

59. The method of claim 58, wherein the input information is dominant-limb information, and the method further comprises:
determining whether the first limb is a left limb or a right limb according to the input information and a determination result.

60. The method of claim 58, wherein the input information indicates whether the first limb is a left limb or a right limb, and the method further comprises:
determining whether the left limb or the right limb of the user is a dominant limb according to the input information and a determination result.

61. A dominant limb identification device, comprising:
a first acquiring module, configured to acquire first somatosensory information of a first limb of a user; and
a first determining module, configured to determine whether the first limb is a dominant limb according to the first somatosensory information and reference information.

62. The device of claim 61, wherein:
the first somatosensory information is first acceleration information;
the first acquiring module is configured to acquire the first acceleration information of the first limb of the user; and
the first determining module is configured to determine whether the first limb is a dominant limb according to the first acceleration information and reference information.

63. The device of claim 62, further comprising:
a second determining module, configured to determine a threshold as the reference information according to an average absolute value of left-limb acceleration information and an average absolute value of right-limb acceleration information of the user.

64. The device of claim 63, wherein the first determining module is configured to:
determine that the first limb is a dominant limb when an average absolute value of the first acceleration information is greater than the threshold; and
determine that the first limb is not a dominant limb when the average absolute value of the first acceleration information is less than the threshold.

65. The device of claim 62, further comprising:
a second acquiring module, configured to acquire second acceleration information of a second limb of the user as the reference information.

66. The device of claim 65, wherein the first determining module is configured to:
determine that the first limb is a dominant limb when an average absolute value of the first acceleration information is greater than an average absolute value of the second acceleration information; and
determine that the first limb is not a dominant limb when the average absolute value of the first acceleration information is less than the average absolute value of the second acceleration information.

67. The device of claim 61, wherein:
the first somatosensory information is first blood flow information;
the first acquiring module is configured to acquire the first blood flow information of the first limb of the user; and
the first determining module is configured to determine whether the first limb is a dominant limb according to the first blood flow information and reference information.

68. The device of claim 67, further comprising:
a second determining module, configured to determine a threshold as the reference information according to an average value of left-limb blood flow information and an average value of right-limb blood flow information of the user.

69. The device of claim 68, wherein the first determining module is configured to:
determine that the first limb is a dominant limb when an average value of the first blood flow information is greater than the threshold; and
determine that the first limb is not a dominant limb when the average value of the first blood flow information is less than the threshold.

70. The device of claim 67, further comprising:
a second acquiring module, configured to acquire second blood flow information of a second limb of the user as the reference information.

71. The device of claim 70, wherein the first determining module is configured to:
determine that the first limb is a dominant limb when an average value of the first blood flow information is greater than an average value of the second blood flow information; and
determine that the first limb is not a dominant limb when the average value of the first blood flow information is less than the average value of the second blood flow information.

72. The device of claim 61, wherein:
the first somatosensory information is first PPG information;
the first acquiring module is configured to acquire the first PPG information of the first limb of the user; and
the first determining module is configured to determine whether the first limb is a dominant limb according to the first PPG information and reference information.

73. The device of claim 72, further comprising:
a second determining module, configured to determine a threshold as the reference information according to an average amplitude value of left-limb PPG information and an average amplitude value of right-limb PPG information of the user.

74. The device of claim 73, wherein the first determining module is configured to:
determine that the first limb is not a dominant limb when an average amplitude value of the first PPG information is greater than the threshold; and
determine that the first limb is a dominant limb when the average amplitude value of the first PPG information is less than the threshold.

75. The device of claim 72, further comprising:
a second acquiring module, configured to acquire second PPG information of a second limb of the user as the reference information.

76. The device of claim 75, wherein the first determining module is configured to:
determine that the first limb is not a dominant limb when an average amplitude value of the first PPG information is greater than an average amplitude value of the second PPG information; and
determine that the first limb is a dominant limb when the average amplitude value of the first PPG information is less than the average amplitude value of the second PPG information.

77. The device of claim 61, wherein:
the first somatosensory information is first temperature information;
the first acquiring module is configured to acquire the first temperature information of the first limb of the user; and
the first determining module is configured to determine whether the first limb is a dominant limb according to the first temperature information and reference information.

78. The device of claim 77, further comprising:
a second determining module, configured to determine a threshold as the reference information according to an average value of left-limb temperature information and an average value of right-limb temperature information of the user.

79. The device of claim 78, wherein the first determining module is configured to:
determine that the first limb is a dominant limb when an average value of the first temperature information is greater than the threshold; and
determine that the first limb is not a dominant limb when the average value of the first temperature information is less than the threshold.

80. The device of claim 77, further comprising:
a second acquiring module, configured to acquire second temperature information of a second limb of the user as the reference information.

81. The device of claim 80, wherein the first determining module is configured to:
determine that the first limb is a dominant limb when an average value of the first temperature information is greater than an average value of the second temperature information; and
determine that the first limb is not a dominant limb when the average value of the first temperature information is less than the average value of the second temperature information.

82. The device of claim 61, wherein:
the first somatosensory information is first humidity information;
the first acquiring module is configured to acquire the first humidity information of the first limb of the user; and
the first determining module is configured to determine whether the first limb is a dominant limb according to the first humidity information and reference information.

83. The device of claim 82, further comprising:
a second determining module, configured to determine a threshold as the reference information according to an average value of left-limb humidity information and an average value of right-limb humidity information of the user.

84. The device of claim 83, wherein the first determining module is configured to:
determine that the first limb is a dominant limb when an average value of the first humidity information is greater than the threshold; and
determine that the first limb is not a dominant limb when the average value of the first humidity information is less than the threshold.

85. The device of claim 82, further comprising:
a second acquiring module, configured to acquire second humidity information of a second limb of the user as the reference information.

86. The device of claim 85, wherein the first determining module is configured to:
determine that the first limb is a dominant limb when an average value of the first humidity information is greater than an average value of the second humidity information; and
determine that the first limb is not a dominant limb when the average value of the first humidity information is less than the average value of the second humidity information.

87. The device of claim 61, wherein:
the first somatosensory information is first fat information;
the first acquiring module is configured to acquire the first fat information of the first limb of the user; and
the first determining module is configured to determine whether the first limb is a dominant limb according to the first fat information and reference information.

88. The device of claim 87, further comprising:
a second determining module, configured to determine a threshold as the reference information according to an average value of left-limb fat information and an average value of right-limb fat information of the user.

89. The device of claim 88, wherein the first determining module is configured to:
determine that the first limb is a dominant limb when an average value of the first fat information is greater than the threshold; and
determine that the first limb is not a dominant limb when the average value of the first fat information is less than the threshold.

90. The device of claim 87, further comprising:
a second acquiring module, configured to acquire second fat information of a second limb of the user as the reference information.

91. The device of claim 90, wherein the first determining module is configured to:
determine that the first limb is a dominant limb when an average value of the first fat information is greater than an average value of the second fat information; and
determine that the first limb is not a dominant limb when the average value of the first fat information is less than the average value of the second fat information.

92. The device of claim 61, wherein:
the first somatosensory information is first alkalinity/acidity information;
the first acquiring module is configured to acquire the first alkalinity/acidity information of the first limb of the user; and
the first determining module is configured to determine whether the first limb is a dominant limb according to the first alkalinity/acidity information and reference information.

93. The device of claim 92, further comprising:
a second determining module, configured to determine a threshold as the reference information according to an average value of left-limb alkalinity/acidity information and an average value of right-limb alkalinity/acidity information of the user.

94. The device of claim 93, wherein the first determining module is configured to:
determine that the first limb is a dominant limb when an average value of the first alkalinity/acidity information is greater than the threshold; and
determine that the first limb is not a dominant limb when the average value of the first alkalinity/acidity information is less than the threshold.

95. The device of claim 92, further comprising:
a second acquiring module, configured to acquire second alkalinity/acidity information of a second limb of the user as the reference information.

96. The device of claim 95, wherein the first determining module is configured to:
determine that the first limb is a dominant limb when an average value of the first alkalinity/acidity information is greater than an average value of the second alkalinity/acidity information; and
determine that the first limb is not a dominant limb when the average value of the first alkalinity/acidity information is less than the average value of the second alkalinity/acidity information.

97. The device of claim 61, wherein:
the first somatosensory information is first skin conductance information;
the first acquiring module is configured to acquire the first skin conductance information of the first limb of the user; and
the first determining module is configured to determine whether the first limb is a dominant limb according to the first skin conductance information and reference information.

98. The device of claim 97, further comprising:
a second determining module, configured to determine a threshold as the reference information according to an average amplitude value of left-limb skin conductance information and an average amplitude value of right-limb skin conductance information of the user.

99. The device of claim 98, wherein the first determining module is configured to:
determine that the first limb is not a dominant limb when an average amplitude value of the first skin conductance information is greater than the threshold; and
determine that the first limb is a dominant limb when the average amplitude value of the first skin conductance information is less than the threshold.

100. The device of claim 97, further comprising:
a second acquiring module, configured to acquire second skin conductance information of a second limb of the user as the reference information.

101. The device of claim 100, wherein the first determining module is configured to:
determine that the first limb is not a dominant limb when an average amplitude value of the first skin conductance information is greater than an average amplitude value of the second skin conductance information; and
determine that the first limb is a dominant limb when the average amplitude value of the first skin conductance information is less than the average amplitude value of the second skin conductance information.

102. The device of claim 61, wherein:
the first somatosensory information is first electromyogram information;
the first acquiring module is configured to acquire the first electromyogram information of the first limb of the user; and
the first determining module is configured to determine whether the first limb is a dominant limb according to the first electromyogram information and reference information.

103. The device of claim 102, further comprising:
a second determining module, configured to determine a threshold as the reference information according to an average amplitude value of left-limb electromyogram information and an average amplitude value of right-limb electromyogram information of the user.

104. The device of claim 103, wherein the first determining module is configured to:
determine that the first limb is a dominant limb when an average amplitude value of the first electromyogram information is greater than the threshold; and
determine that the first limb is not a dominant limb when the average amplitude value of the first electromyogram information is less than the threshold.

105. The device of claim 102, further comprising:
a second acquiring module, configured to acquire second electromyogram information of a second limb of the user as the reference information.

106. The device of claim 105, wherein the first determining module is configured to:
determine that the first limb is a dominant limb when an average amplitude value of the first electromyogram information is greater than an average amplitude value of the second electromyogram information; and
determine that the first limb is not a dominant limb when the average amplitude value of the first electromyogram information is less than the average amplitude value of the second electromyogram information.

107. The device of claim 61, wherein:
the first somatosensory information is first bio-impedance information;
the first acquiring module is configured to acquire the first bio-impedance information of the first limb of the user; and
the first determining module is configured to determine whether the first limb is a dominant limb according to the first bio-impedance information and reference information.

108. The device of claim 107, further comprising:
a second determining module, configured to determine a threshold as the reference information according to an average amplitude value of left-limb bio-impedance information and an average amplitude value of right-limb bio-impedance information of the user.

109. The device of claim 108, wherein the first determining module is configured to:
determine that the first limb is a dominant limb when an average amplitude value of the first bio-impedance information is greater than the threshold; and
determine that the first limb is not a dominant limb when the average amplitude value of the first bio-impedance information is less than the threshold.

110. The device of claim 107, further comprising:
a second acquiring module, configured to acquire second bio-impedance information of a second limb of the user as the reference information.

111. The device of claim 110, wherein the first determining module is configured to:
determine that the first limb is a dominant limb when an average amplitude value of the first bio-impedance information is greater than an average amplitude value of the second bio-impedance information; and
determine that the first limb is not a dominant limb when the average amplitude value of the first bio-impedance information is less than the average amplitude value of the second bio-impedance information.

112. The device of claim 61, wherein:
the first somatosensory information is first blood oxygen information;
the first acquiring module is configured to acquire the first blood oxygen information of the first limb of the user; and
the first determining module is configured to determine whether the first limb is a dominant limb according to the first blood oxygen information and reference information.

113. The device of claim 112, further comprising:
a second determining module, configured to determine a threshold as the reference information according to an average value of left-limb blood oxygen information and an average value of right-limb blood oxygen information of the user.

114. The device of claim 113, wherein the first determining module is configured to:
determine that the first limb is not a dominant limb when an average value of the first blood oxygen information is greater than the threshold; and
determine that the first limb is a dominant limb when the average value of the first blood oxygen information is less than the threshold.

115. The device of claim 112, further comprising:
a second acquiring module, configured to acquire second blood oxygen information of a second limb of the user as the reference information.

116. The device of claim 115, wherein the first determining module is configured to:
determine that the first limb is not a dominant limb when an average value of the first blood oxygen information is greater than an average value of the second blood oxygen information; and
determine that the first limb is a dominant limb when the average value of the first blood oxygen information is less than the average value of the second blood oxygen information.

117. The device of claim 61, further comprising:
an execution module, configured to perform an operation according to a determination result.

118. The device of claim 61, further comprising:
an input module, configured to receive input information from the user.

119. The device of claim 118, wherein the input information is dominant-limb information, and the device further comprises:
a second determining module, configured to determine whether the first limb is a left limb or a right limb according to the input information and a determination result.

120. The device of claim 118, wherein the input information comprises whether the first limb is a left hand or a right hand, and the device further comprises:
a third determining module, configured to determine whether the user is left-handed or right-handed according to the input information and a determination result.

121. The device of claim 61, wherein the device is a wearable device.

122. A computer readable storage medium, comprising at least one executable instruction, which, when executed by a processor of a dominant limb identification device, causes the processor to perform operations comprising:
acquiring first somatosensory information of a first limb of a user; and
determining whether the first limb is a dominant limb according to the first somatosensory information and reference information.

123. A dominant limb identification device, characterized by comprising a processor and a memory, the memory storing computer executable instructions, the processor being coupled to the memory through a communication bus, and when the dominant limb identification device operates, the processor executes the computer executable instructions stored in the memory, causing the dominant limb identification device perform operations comprising:
acquiring first somatosensory information of a first limb of a user; and
determining whether the first limb is a dominant limb according to the first somatosensory information and reference information.
